(19) United States
(12) Patent Application Publication
(10) Pub. No.: US 2019/0086390 A1
(43) Pub. Date: Mar. 21, 2019

(54) INDIRECT ASSESSMENT OF INSULIN RELEASE IN A CELL

(71) Applicant: Board of Supervisors of Louisiana State University and Agricultural and Mechanical College, Baton Rouge, LA (US)

(72) Inventors: RICHARD C. ROGERS, CLINTON, LA (US); JASON COLLIER, DENHAM SPRINGS, LA (US)

(73) Assignee: Board of Supervisors of Louisiana State University and Agricultural and Mechanical College, Baton Rouge, LA (US)

(21) Appl. No.: 16/080,800

(22) PCT Filed: Mar. 1, 2017
(86) PCT No.: PCT/US17/20137

§ 371 Date: Aug. 29, 2018

Related U.S. Application Data
(60) Provisional application No. 62/301,714, filed on Mar. 1, 2016.

(51) Int. Cl. G01N 33/50; A61P 3/08; G01N 33/487; G01N 33/52; G01N 33/74

(52) U.S. Cl. CPC G01N 33/507; A61P 3/08; G01N 33/487; G01N 2800/042; G01N 33/5035; G01N 33/74; G01N 33/52

(57) ABSTRACT

Provided herein are compositions, systems, kits, and methods of indirectly assessing insulin release in a cell.

### CROSS-REFERENCE TO RELATED APPLICATIONS

This application claims the benefit of and priority to co-pending U.S. Provisional Patent Application No. 62/301,714, filed on Mar. 1, 2016, entitled “INDIRECT ASSESSMENT OF INSULIN RELEASE IN A CELL,” the contents of which is incorporated by reference herein in its entirety.

### STATEMENT REGARDING FEDERALLY SPONSORED RESEARCH OR DEVELOPMENT

This invention was made with government support under grant number P30 GM118430 and P50 AT002776 awarded by the National Institutes of Health. The government has certain rights to the invention.

### BACKGROUND

Almost 10% of the population in the United States has diabetes, with about 1.7 million new cases diagnosed in 2012. Moreover 86 million Americans are considered to be pre-diabetic. Diabetes remains the seventh leading cause of death in the United States and was a contributing factor in the death of almost a quarter million Americans in 2010. As such, there exists a need for improved assays, compounds, and treatments to reduce and control the impact of diabetes on the health of Americans.

### SUMMARY

Provided herein are methods to indirectly assess insulin release in a cell that can include the steps of culturing cells, incubating the cells with a calcium sensitive indicator for an amount of time, stimulating the cells with glucose, and measuring a signal from the calcium sensitive indicator. The cells can be pancreatic beta cells. The method can further include the step of contacting the cells with a composition. The composition can be a pharmaceutical composition. The composition can be a chemical compound. The composition can be a biologic compound. The pancreatic beta cells can be obtained from a subject in need thereof. The subject in need thereof can have diabetes or a symptom thereof. The calcium sensitive indicator can be a fluorescent indicator. The calcium sensitive indicator can be selected from the group of: fura-2, indo-1, fluo-3, fluo-4, calcium green-1, and a calcium binding fluorescent protein. The step of measuring a signal can include the step of microscopically visualizing the pancreatic beta cells. The step of measuring a signal can further include detecting a wavelength of light emitted by the calcium sensitive indicator. In some aspects, the step of measuring a signal can include detecting a wavelength of light emitted by the calcium sensitive indicator. The wavelength of light can be detected by a plate reader. In some aspects, during the step of incubating the cells with a calcium sensitive indicator for an amount of time, the cells can also be incubated with a surfactant.

Also provided herein are kits that can include a cell culture plate having a well, pancreatic cells, where the pancreatic cells are contained in the well, and a calcium sensitive indicator. The calcium sensitive indicator can be selected from the group of: fura-2, indo-1, fluo-3, fluo-4, calcium green-1, and a calcium binding fluorescent protein. The calcium sensitive indicator can be contained in the well. The calcium sensitive indicator can be contained in the pancreatic cells. The calcium sensitive indicator can be a calcium binding fluorescent protein.

### BRIEF DESCRIPTION OF THE DRAWINGS

Further aspects of the present disclosure will be readily appreciated upon review of the detailed description of its various embodiments, described below, when taken in conjunction with the accompanying drawings.

FIG. 1 shows embodiments of a method of indirect assessment of insulin release in a cell. Cultured rat or human pancreatic beta cells can be grown in (for example) 96-well glass bottomed culture plates. They can be sealed and shipped to the user. Fluorescent calcium reporter dyes and a surfactant can be added to the wells. After about 30-60 minutes, the dye can be washed out and replaced with an experimental buffer. Cells can respond to increasing concentrations of glucose by releasing stored calcium in proportion to glucose signal. Insulin can be secreted in proportion to the intracellular calcium signal. The calcium signal can be reported by calcium sensitive dye fluorescence. In this assay, calcium fluorescence can be used a linear proxy for insulin secretion. The effects of libraries of chemical agents on the ability of beta cells to secrete insulin can be screened much faster and at much less expense than with typical ELISA or RIA methods. FIGS. 2A-5 demonstrate validation of the assay.

FIGS. 2A-2C show fluorescent microscopic images of cultured pancreatic β-cells pre-labeled with a calcium fluorescent reporter dye (e.g. Calcium Green). Areas 1, 2, and 3 outline three cells as discrete areas of interest to be monitored for their response to change in glucose concentration from a hypoglycemic state (2 mM glucose, 36 mg %) to a hyperglycemic state (12 mM, 216%) and back.

FIG. 3 shows a graph demonstrating calcium dye fluorescence measured in the three areas highlighted in FIGS. 2A-2C and demonstrating the individual β-cell calcium response to the glucose pulse.

FIG. 4 shows a graph demonstrating insulin secretion measured using a traditional radioimmunoassay (RIA) methods from the same cell culture in response to the same pulse of glucose as demonstrated in FIGS. 2A-2C.

FIG. 5 shows a graph demonstrating secretion of insulin (solid line) or CCL2 (dashed line) into the culture media of 832/13 cells stimulated with 1 ng/ml IL-1β for the indicated times as quantified by enzyme linked immunosorbent assay (ELISA). ***p<0.001 vs. untreated (NT; black bars). Data are presented as means±SE of 3 independent experiments. **P<0.01; *P<0.05. NS, not significant.

FIG. 6 shows a graph demonstrating Relative mRNA abundance of inducible nitric oxide synthase (iNOS) as quantified by RT-PCR, with protein abundance determined by immuno-blotting (inset) in 832/13 cells stimulated with 1 ng/ml IL-1β for the indicated times.

FIG. 7 shows a graph demonstrating nitrite accumulation in the culture media of 832/13 treated with 1 ng/ml IL-1β for the indicated times as assessed using the Griess assay. ***p<0.001 vs. untreated (NT; black bars). Data are presented as means±SE of 3 independent experiments. **P<0.01; *P<0.05. NS, not significant.

FIG. 8 shows a graph demonstrating insulin secretion was measured in response to basal (2 mM) or stimulatory (16 mM) glucose concentrations in 832/13 and 833/15 insulinoma cells that were either untreated (control) or stimulated with both 1 ng/ml IL-13 and 100 U/ml IFN-γ for 18 h. ***p<0.001 vs. untreated (NT; black bars). Data are presented as means±SE of 3 independent experiments. **P<0.01; *P<0.05. NS, not significant.

FIG. 9 shows a graph demonstrating insulin secretion during static incubation was measured and represented as percent of the stimulated control in isolated rat islets treated with AdCMV-fS-galactosidase (β-Gal) or AdCMV-IKBa super-repressor (IkBo(^{sr}) in the presence or absence of 1 ng/ml IL-13 and 100 U/ml IFN-y. ***p<0.001 vs. untreated (NT; black bars). Data are presented as means±SE of 3 independent experiments. **P<0.01; *P<0.05. NS, not significant.

FIG. 10. shows a graph demonstrating an immunoblot analysis of whole cell lysates probed with antibodies against iNOS, iKBa, and tubulin. Solid arrow, adenovirally overexpressed IkBα; dashed arrow, endogenous IkBα. 832/13 cells were transduced with adenoviruses expressing either AdCMV-p-Gal [control (C)[ or increasing doses of AdCMV-IKBa^{SR} (low and high). Eight hours after adenoviral transduction, cells were then NT or stimulated for an additional 18 h with 1 ng/ml IL-1β. The high dose of AdCMV-IKBa^{SR} was used in H. *P<0.05 vs. β-Gal-IL-1β; **P<0.01 vs. fi-Gal-IL-1β; #P<0.1 vs. (β-Gal-IL-1β. Data represent means±SE of 3 independent experiments.

FIG. 11 shows a graph demonstrating the results from a densitometric analysis of iNOS protein abundance from the immunoblot shown in FIG. 10. The high dose of AdCMV-IKBa^{SR} was used in H. *P<0.05 vs. β-Gal-IL-1β; **P<0.01 vs. β-Gal-IL-1β; #P<0.1 vs. (β-Gal-IL-1β. Data represent means±SE of 3 independent experiments.

FIG. 12 shows a graph demonstrating the results from a densitometric analysis of IkBoi protein abundance from the immunoblot shown in FIG. 10. The high dose of AdCMV-IKBa^{SR} was used in H. *P<0.05 vs. β-Gal-IL-1β; **P<0.01 vs. β-Gal-IL-1β; #P<0.1 vs. (β-Gal-IL-1β. Data represent means±SE of 3 independent experiments.

FIG. 13 shows a graph demonstrating nitric oxide determination by electron paramagnetic resonance (EPR) spectroscopy combined with A′-methyl-d-glucamine dithiocarbaniate (MGD)₂Fe²⁺ spin trap. 832/13 cells were transduced with adenoviruses expressing either AdCMV-p-Gal [control (C)] or increasing doses of AdCMV-IKBa^{SR} (low and high). Eight hours after adenoviral transduction, cells were then NT or stimulated for an additional 18 h with 1 ng/ml IL-1β. The high dose of AdCMV-IKBa^{SR} was used in H. *P<0.05 vs. β-Gal-IL-1β; *P<0.01 vs. β-Gal-IL-1β; #P<0.1 vs. β-Gal-IL-1β. Data represent means±SE of 3 independent experiments.

FIG. 14 shows a graph demonstrating the quantification of the EPR spectroscopy data shown in FIG. 13. 832/13 cells were transduced with adenoviruses expressing either AdCMV-p-Gal [control (C)] or increasing doses of AdCMV-IKBa^{SR} (low and high). Eight hours after adenoviral transduction, cells were then NT or stimulated for an additional 18 h with 1 ng/ml IL-1β. The high dose of AdCMV-IKBa^{SR} was used in H. *P<0.05 vs. β-Gal-IL-1β; **P<0.01 vs. β-Gal-IL-1β; #P<0.1 vs. (β-Gal-IL-1β. Data represent means±SE of 3 independent experiments.

FIG. 15 shows a graph demonstrating nitrite accumulation in the media was measured by Griess assay. 832/13 cells were transduced with adenoviruses expressing either AdCMV-p-Gal [control (C)] or increasing doses of AdCMV-IKBa^{SR} (low and high). Eight hours after adenoviral transduction, cells were then NT or stimulated for an additional 18 h with 1 ng/ml IL-1β. The high dose of AdCMV-IKBa^{SR} was used in H. *P<0.05 vs. β-Gal-IL-1β; *P<0.01 vs. β-Gal-IL-1β; #P<0.1 vs. β-Gal-IL-1β. Data represent means±SE of 3 independent experiments.

FIG. 16 shows a graph demonstrating insulin production in 832/13 cells were cultured with the indicated adenoviruses for 8 h, followed by exposure to 1 ng/ml IL-1 for 6 h with data shown as % stimulated control. The high dose of AdCMV-IKBa^{SR} was used in H. *P<0.05 vs. β-Gal-IL-1β; **P<0.01 vs. fi-Gal-IL-ip; #P<0.1 vs. β-Gal-IL-1β. Data represent means±SE of 3 independent experiments.

FIG. 17 shows a graph demonstrating insulin production in 832/13 cells were cultured with the indicated adenoviruses for 8 h, followed by exposure to 1 ng/ml IL-1β for 8 h with data shown as % stimulated control. The high dose of AdCMV-IKBa^{SR} was used in H. *P<0.05 vs. β-Gal-IL-1β; **P<0.01 vs. fi-Gal-IL-1β; #P<0.1 vs. β-Gal-IL-1β. Data represent means±SE of 3 independent experiments. Data represent means±SE of a minimum of 3 independent experiments. **P<0.01 vs. lean (fa/+) control; ***P<0.001 vs. β-Gal-NT.

FIGS. 18A-18B shows graphs demonstrating relative mRNA abundance of IL-1 (FIG. 18A) and Nkx6.1 (FIG. 18B) in islets isolated from 8-wk-old lean (fa/+) and obese (fa/fa) Zucker diabetic fatty (ZDF) rats (n=8 rats/group) as measured by RT-PCR measurement expressed as fold over lean control. Data represent means±SE of a minimum of 3 independent experiments. **P<0.01 vs. lean (fa/+) control; ***P<0.001 vs. β-Gal-NT.

FIG. 19 shows an image of an immunoblot of cytosolic and nuclear fractions probed with antibodies directed against iNOS, NKx6.1, Pdx-1, and tubulin. The cytosolic and nuclear fractions are from 832/13 cells transduced with AdCMV-p-Gal or the high dose of AdCMV-IkBα^{sr}. Following an overnight incubation with the indicated adenoviruses, cells were either left untreated or exposed to 1 ng/ml IL-1β for 4 h. Data represents 2 independent experiments. **P<0.01 vs. lean (fa/+) control; ***P<0.001 vs. p-Gal-NT.

FIG. 20 shows a graph demonstrating densitometric analysis of Nkx6.1 protein abundance normalized to the abundance of tubulin of FIG. 19. Data represent means±SE of a minimum of 3 independent experiments. **P<0.01 vs. lean (fa/+) control; ***P<0.001 vs. β-Gal-NT.

FIG. 21 shows a graph demonstrating Tritiated thymidine incorporation into DNA of 832/13 cells transduced with AdCMV-p-Gal or the high dose of AdCMVTkBa^{SR} for 8 h, followed by 18 h of exposure to 1 ng/ml IL-1β. Tritiated thymidine incorporation into DNA was measured at the end of the 18-h cytokine exposure. Data represent means±SE of a minimum of 3 independent experiments. *P<0.01 vs. lean (fa/+) control; ***P<0.001 vs. β-Gal-NT.

FIG. 22 shows a graph demonstrating nitrite (as expressed as fold over control) in the culture media of 832/13 cells were co-treated with 1 ng/ml IL-1β and 1 mM N^{G}-monomethyl-L-arginine (L-NMMA) for 18 h using the Griess method. Data are presented as means±SE of 3 independent experiments. ***/><0.001; **P<0.01; *P<0.05.

FIG. 23 shows a graph demonstrating nitric oxide in 832/13 cells were co-treated with 1 ng/ml IL-1β and 1 mM N^{G}-monomethyl-L-arginine (L-NMMA) for 18 h as measured by EPR spectroscopy combined with (MGD)₂Fe²⁺ spin trapping. Data are presented as means±SE of 3 independent experiments. ***/><0.001; **P<0.01; *P<0.05.

FIG. 24 shows a graph demonstrating EPR spectroscopy combined with (MGD)₂Fe²⁺ spin trapping of 832/13 cells were co-treated with 1 ng/ml IL-1β and 1 mM N^{G}-monomethyl-L-arginine (L-NMMA) for 18 h. Data are presented as means±SE of 3 independent experiments. ***/><0.001; **P<0.01; *P<0.05.

FIG. 25 shows a graph demonstrating insulin secretion in 832/13 cells were co-treated with 1 ng/ml IL-1β and 1 mM N^{G}-monomethyl-L-arginine (L-NMMA) for 18 h as measured in response to basal (2 mM) or stimulatory (16 mM) glucose concentrations. Data are presented as means±SE of 3 independent experiments. ***/><0.001; **P<0.01; *P<0.05.

FIG. 26 shows a graph demonstrating glucose-stimulated insulin secretion 832/13 cells were NT or exposed to 1 ng/ml IL-1β for 18 h as measured in response to basal (2 mM) or stimulatory (16 mM) glucose concentrations in the presence or absence of 10 uM dimethylmalate (DMM), with DMM added only during the final 2 h of the secretion experiment. Data are presented as means±SE of 3 independent experiments. ***/><0.001; **P<0.01; *P<0.05.

FIGS. 27A-27D show graphs demonstrating mitochondrial number as determined by using the ratio of mitochondrial DNA (COI or ATP6) relative to nuclear DNA (NDUFA or SDHA) in 832/13 and 833/15 cells were NT or treated with 1 ng/ml IL-1β alone or IL-1β+100 U/mL IFNy for either 6 or 18 h. Values represent means±SE of 3 independent experiments. **P<0.01; *P<0.05.

FIG. 28 shows a graph demonstrating the cellular ability of 832/13 cells exposed to 1 ng/ml IL-1β in the presence or absence of 1 mM I-NMMA for 18 h to reduce the MTS [3-(4,5-dim-ethylthiazol-2-yl)-5-(3-carboxymethoxyphe-nyl)-2-(4-sulfophenyl)-2H-tetrazolium, inner salt] dye. Values represent means±SE of 3 independent experiments. **P<0.01; *P<0.05.

FIG. 29 shows a graph demonstrating the oxygen consumption rate (OCR) of 832/13 cells exposed to 1 ng/ml IL-ip in the presence or absence of 1 mM I-NMMA for 18 h as calculated by an area under the curve (AUC) analysis. Values represent means±SE of 3 independent experiments. **P<0.01; *P<0.05.

FIG. 30 shows a graph demonstrating the glucose-induced calcium response (as expressed as % change in fluorescence) in 832/13 cells were co-treated for 18 h with 1 ng/ml IL-1β and 1 mM I-NMMA, followed by a bath-applied glucose challenge to evoke elevations in intracellular calcium levels as measured by changes in fluorescence. *P<0.05.

FIGS. 31A-31B show graphs demonstrating whole cell voltage clamp recordings from a control and an IL-1β-treated cell. Cells were held at −70 mV and then stepped for 300 ms to a series of voltages ranging from −103 mV to +17 mV. Shown are the currents generated by the step to −3 mV. Voltage step values are corrected for the liquid junction potential (−13 mV).

FIG. 32 shows a graph demonstrating the mean current densities (voltage step to −3 mV) for control and IL-1β-treated cells. No significant difference in current amplitude was observed (n=25 for both).

FIG. 33 shows a graph demonstrating the measured capacitance values for the 2 groups of cells indicate that cell size for IL-I1β-treated cells was also not significantly different from control (n=25 for both).

FIGS. 34A-34B show graphs demonstrating CCL2 release (FIG. 34A) and CCL20 release (FIG. 34B) in 832/13 cells co-treated for 6 h with 1 ng/ml IL-1β and 1 mM I-NMMA as measured by ELISA. Data represent means±SE of a minimum of 3 independent experiments. **P<0.001 vs. β-Gal-IL-1β; **P<0.01 vs. β-Gal-IL-1β.

FIGS. 35A-35B show graphs demonstrating CCL2 release (FIG. 35A) and CCL20 release (FIG. 35B) in 832/13 cells were transduced with adenoviruses overexpress-ing either AdCMV-p-Gal or increasing doses of AdCMV-IKBct^{SR} (low and high). Twelve hours after adenoviral transduction, cells were then NT or exposed to 1 ng/ml IL-1β for an additional 6 h as measured by ELISA. Data represent means±SE of a minimum of 3 independent experiments. ***P<0.001 vs. β-Gal-IL-1β; **P<0.01 vs. β-Gal-IL-1β.

FIGS. 36A-36B show graphs demonstrating CCL2 release (FIG. 36A) and CCL20 release (FIG. 36B) in 832/13 cells were NT or exposed to 1 ng/ml IL-ip for 6 h in the presence of increasing concentrations of glucose (2, 4, 8, or 16 mM) as measured by ELISA. Data represent means±SE of a minimum of 3 independent experiments. ***P<0.001 vs. β-Gal-IL-1P; **P<0.01 vs. β-Gal-IL-1β.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed. As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of cell biology, molecular biology, microbiology, nanotechnology, organic chemistry, biochemistry, endocrinology and the like, which are within the skill of the art. Such techniques are explained fully in the literature.

### Definitions

Unless otherwise defined herein, terms will have their ordinary and customary meaning as they would be appreciated by one of ordinary skill in the art in view of this disclosure.

As used herein, “about,” “approximately,” and the like, when used in connection with a numerical variable, can generally refer to the value of the variable and to all values of the variable that are within the experimental error (e.g., within the 95% confidence interval for the mean) or within ±10% of the indicated value, whichever is greater.

As used herein, “calcium sensitive” can refer to agents taken up by live cells that report changes in intracellular calcium by changing their fluorescence.

As used herein, “cell,” “cell line,” and “cell culture” include any progeny. It is also understood that all progeny may not be precisely identical in DNA content, due to deliberate or inadvertent mutations. Variant progeny that have the same function or biological property, as screened for in the originally transformed cell, are included.

As used herein, “control” is an alternative subject or sample used in an experiment for comparison purposes and included to minimize or distinguish the effect of variables other than an independent variable.

As used herein, “culturing” can refer to maintaining cells under conditions in which they can proliferate and avoid senescence as a group of cells. “Culturing” can also include conditions in which the cells also or alternatively differentiate.

As used herein, “glucose responsive” can refer to a cell's, or population thereof, ability to alter intracellular calcium content in proportion to the extracellular concentration of glucose.

As used herein, “positive control” can refer to a “control” that is designed to produce the desired result, provided that all reagents are functioning properly and that the experiment is properly conducted.

As used herein, “negative control” can refer to a “control” that is designed to produce no effect or result, provided that all reagents are functioning properly and that the experiment is properly conducted. Other terms that are interchangeable with “negative control” include “sham,” “placebo,” and “mock.”

Discussion

Almost 10% of the population in the United States has diabetes, with about 1.7 million new cases diagnosed in 2012. Moreover 86 million Americans are considered to be pre-diabetic. Diabetes remains the seventh leading cause of death in the United States and was a contributing factor in the death of almost a quarter million Americans in 2010. There have been great efforts to develop treatments, including small molecules and biologics, to combat this disease. Further, personalized medicine affords the opportunity to understand a disease at the individual patient level. Cell based assays are a workhorse of efforts to develop treatments and offer personalized medicine approaches to disease diagnosis and management to patients.

Most cell-based assays available today are designed to determine the concentrations of cellular products such as hormones, cytokines, or neurotransmitters. Such assays are also used to assess toxicity of different reagents by monitoring cell death. At present, there are no high-throughput assays of the glucodetection-insulin secretion mechanism of pancreatic beta cells.

With that said, described herein are assays where pancreatic beta cells can be preloaded with a calcium-sensitive indicator, the pancreatic beta cells can be stimulated with a glucose challenge, a signal from the calcium-sensitive indicator can be measured. Optionally, the insulin secretion from the cell can be determined from the measured signal from the calcium-sensitive indicator. The compositions, systems, and methods described herein do not rely on the time consuming and expensive processes of actually measuring insulin secretion with radio immunoassays (RIA) or enzyme-linked immunosorbant assay (ELISA). Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

Assays

Insulin secretion by the beta cell is preceded by intracellular release of calcium from the endoplasmic reticulum (ER) and the magnitude of the calcium release is proportional to the insulin release. Therefore, by measuring the release of calcium in beta cells in response to a stimulus, such as glucose, can be used to indirectly evaluate the ability of cells to secrete insulin in response to a stimulus.

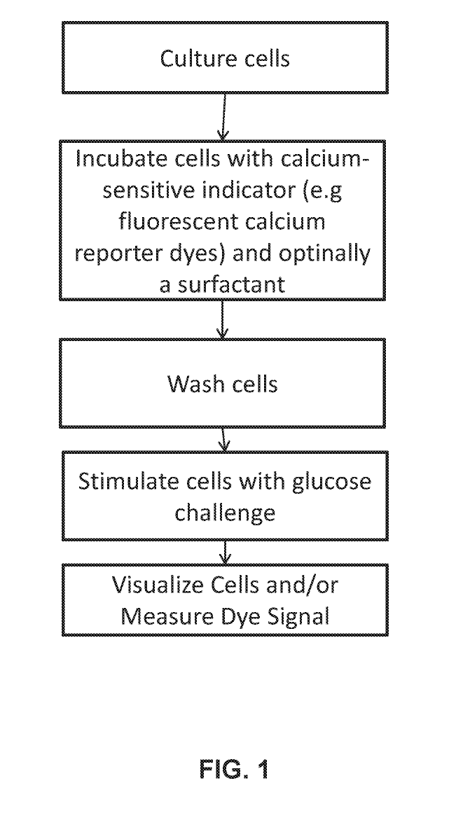

With that in mind, provided herein are assays that can detect glucodetection-insulin secretion mechanism of a cell(s). Discussion of the methods begins with FIG. 1, which shows embodiments of a method that can include the steps of culturing cells, incubating the cells for an amount of time with a calcium sensitive indicator, stimulating the cells with glucose, and measuring a signal from the calcium sensitive indicator. In some embodiments, the cells can be glucose responsive cells. The glucose responsive cells can be pancreatic beta cells.

The methods can further include the step of contacting the cells with a composition. The composition can be a pharmaceutical composition. The composition can be a chemical compound. The composition can be a biologic compound. Exemplary compositions can include, but are not limited to, candidate and/or experimental drug compounds, acarbose, albiglutide, alogliptin, metformin, pioglitazone, bromocriptine mesylate, canaglifozin, dapagliflozin, dulaglutide, empagliflozin, linagliptin, exenatide, glimepiride, glyburide, glipizide linagliptin, liraglutide, miglitol, nateglinide, repaglinide, rosiglitazone, saxagliptin, sitagliptin and combinations thereof. Botanical extracts from, for example, members of the Artemesia and Botanical extracts of the Helenium species have been associated with anti-diabetic effects and could be rapidly screened with this assay. That is, pretreatment of the cells in culture before the assay can be done to assess the effect of these botanical extracts on the glucose sensitivity of the cells.

The cells, such as pancreatic cells, can be from a subject in need thereof. The subject in need thereof can have diabetes or be in a pre-diabetic state according to a physician's diagnosis. The subject in need thereof can be receiving a treatment, such as a pharmaceutical composition, for diabetes.

The calcium sensitive indicator can be a fluorescent indicator. Suitable calcium sensitive indicators can include, but are not limited to, fura-2, indo-1, fluo-3, fluo-4, calcium green-1, and a calcium binding fluorescent protein. In some embodiments, the cells can transiently or stably express a calcium binding fluorescent protein. In some embodiments, the cells can contain a nucleic acid vector that can contain a nucleic acid that can be transcribed into a calcium binding fluorescent protein. The amount of time the cells are incubated with the calcium sensitive indicator can range from 30 minutes to an hour or more. In some aspects, the cells can be incubated with a calcium sensitive indicator and a surfactant. Suitable surfactants include, but are not limited to pluronic acid in DMSO. The concentration of surfactant can range from 0.01 to 0.05%.

The cells can be stimulated by contacting the cells with an amount or a concentration of glucose for an amount of time. The concentration of glucose can range from 0, 1, 2, 3, 4, 5, 6, 7, 8, 9, 10, 11, 12, 13, 14 mM to 15 mM or more. In some embodiments the concentration of glucose is about 2 mM. In some embodiments the concentration of glucose can be about 12 mM. The glucose can be added as a single dose, over multiple doses. In further embodiments, an increasing or decreasing concentration can be provided to the cells over a time period by changing the glucose concentration of a solution that can be continuously perfused over the cells. The time in that the cells are in contact with the glucose can range from about 2 to about 10 minutes or more. The step of measuring the signal from the calcium sensitive indicator can include microscopically visualizing the cells. The cells can be on coverslips or microscope slides. In other embodiments, the step of measuring the signal from the calcium sensitive indicator can include detecting a wavelength of light emitted by the calcium sensitive indicator. The wavelength of light can be detected by a device that can be configured to detect a wavelength of light. The wavelength of light can range from about 350 nm to about 750 nm and can be any integer or range therein. In some embodiments, the device can be a device configured to take readings from individual wells on a cell culture plate (e.g. a plate reader or other suitable device configured to detect light from a cell or population thereof).

The method can further include the step of calculating the timing and/or an amount (such as a relative amount) of insulin secretion by a cell in response to the stimulus, including a glucose stimulus. For example, a 2-12 mM step increase produces a 2-8uU increase in insulin production and a corresponding 30% increase in calcium fluorescence.

Kits

Also provided herein are kits including cells and a calcium indicator contained in a cell culture plate or other cell culture container. The kits can provide a ready-to-use device for drug screening assays. In some embodiments the kits can include a cell culture plate having a well, cells, where the cells are contained within the well, and a calcium sensitive indicator. In some embodiments, the cells are glucose responsive cells. The glucose responsive cells can be pancreatic beta cells. In some embodiments, the cells are seeded on glass slides or coverslips that are contained within the well. In other embodiments, the cells are seeded directly onto the surface of the well, on a feeder layer of cells in the well, or on a synthetic or natural matrix layer disposed of in the cell. Suitable cell culture dishes, plates, or other containers are generally known in the art. Suitable feeder cells are generally known in the art. Suitable synthetic or natural matrices for cell culture are generally known in the art. Examples include, but are not limited to, poly-lysine and collagen.

The kit can further include a cell culture media. The cell culture media can be contained in the well. The cells can be contained in the cell culture media within the well. Suitable cell culture media are generally known in the art.

The calcium sensitive indicator can be a fluorescent indicator. Suitable calcium sensitive indicators can include, but are not limited to, fura-2, indo-1, fluo-3, fluo-4, calcium green-1, and a calcium binding fluorescent protein. In some embodiments, the cells can transiently or stably express a calcium binding fluorescent protein. In some embodiments, the cells can contain a nucleic acid vector that can contain a nucleic acid that can be transcribed into a calcium binding fluorescent protein. In some embodiments, the calcium sensitive indicator is contained in the cell media within the well. In other embodiments, the calcium sensitive indicator is provided in a separate container from the cell culture plate. In some embodiments, the cells contain the calcium sensitive indicator. In some embodiments, the cells are transformed, transduced, or transfected with nucleic acid vector, where the nucleic acid can encode a calcium binding fluorescent protein. The cells can contain a calcium sensitive fluorescent protein.

The kit can contain other reagents, solutions, vials, syringes, and other implementations that can be used in the preparation or execution of any of the methods, devices, or compositions described herein.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure.

### Example 1

Beta cell secretion of insulin is dependent on the intracellular release of calcium from storage organelles within a cell (e.g. the endoplasmic reticulum (ER)). Insulin secretion by the beta cells is preceded by an intracellular release of calcium from the ER. As demonstrated herein the magnitude of the intracellular calcium release in the cell is proportional to the insulin release. As demonstrated in this Example, specific combinations of cell culture techniques and calcium imaging techniques can allow for evaluation of the ability of isolated beta cells to release insulin.

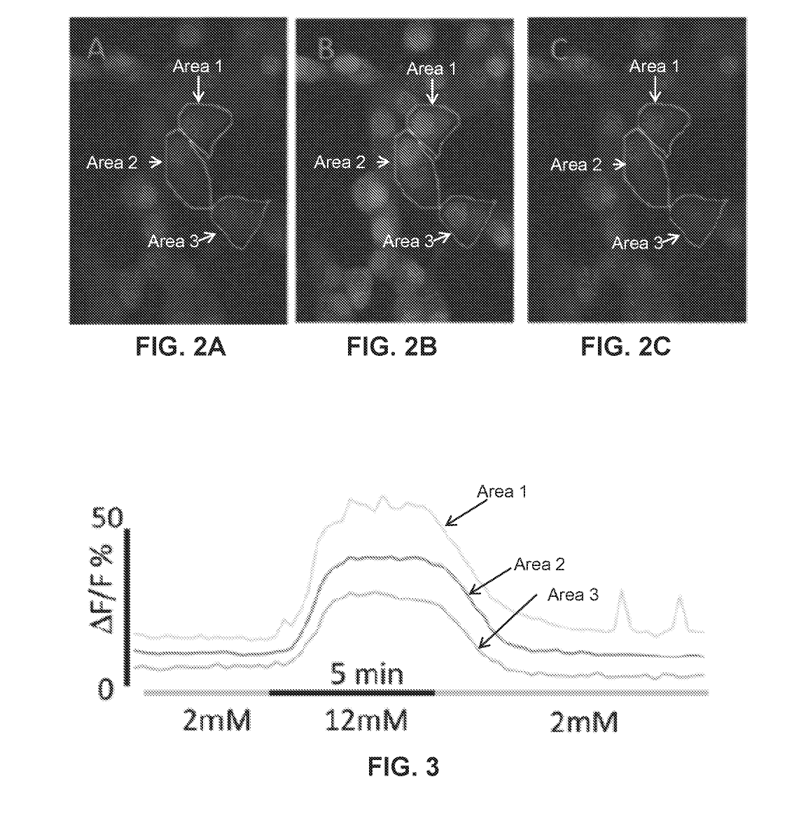

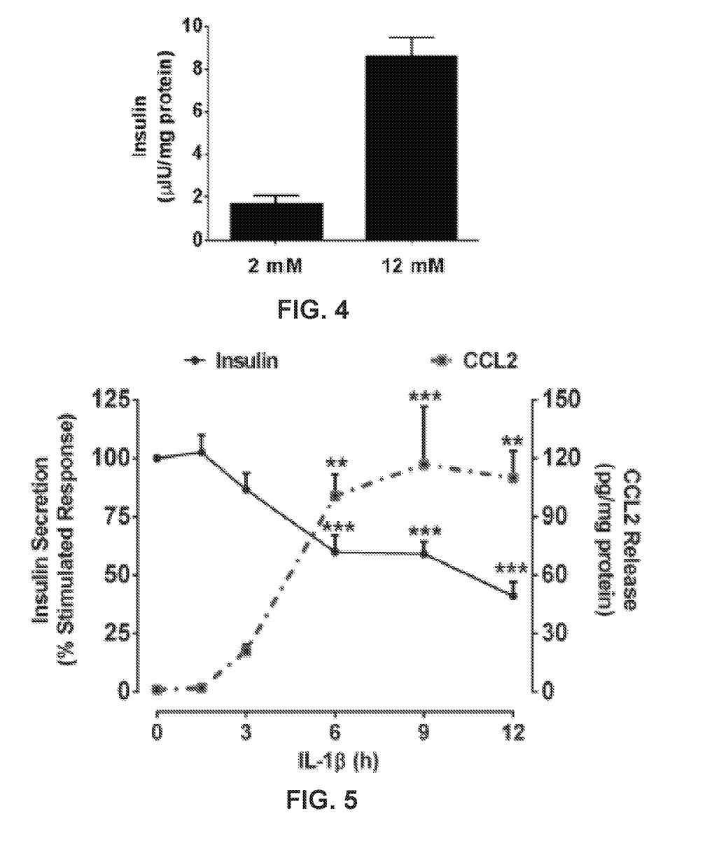

As demonstrated in FIGS. 2A-2C demonstrate beta cells pre-labeled with the fluorescent reporter dye Calcium Green. As an example, three cells (Areas 1, 2, and 3) are outlined in FIGS. 2A-2C as discrete “regions of interest” to be monitored for their response to change in glucose concentration from 2 mM (36 mg % or “hypoglycemic”) to 12 mM (216 mg % or “hyperglyemic”) and back. An sharp increase in intracellular calcium in response to the high glucose challenge that returns to baseline with a reduction in glucose to “hypoglycemic” levels was observed as demonstrated by the change in fluorescence in the three areas identified in FIGS. 2A-2C and 3. The results of the fluorescent based assay were compared to a traditional radioimmunoassay. Insulin secretion measured using radioimmunoassay methods from the same cell culture as used for the fluorescent based insulin secretion assay in response to the same pulse of glucose as demonstrated in FIGS. 2A-2C and 3.

### Example 2

The progression to both type 1 (T1DM) and type 2 diabetes mellitus (T2DM) proceeds via immune cell-associated alterations in islet p-cell mass and function. Alterations in islet P-cell mass and function are two major determinants controlling the total amount of insulin produced and secreted in response to physiological stimuli (e.g., glucose). Proinflammatory cytokines such as IL-1β and IFNy contribute significantly to losses in islet β-cell viability and insulin secretion. Islet β-cell exposed to IL-1β and IFNy undergo extensive genetic reprogramming, which includes transcriptional increases in the inducible nitric oxide synthase (iNOS) gene (13, 26) and various genes encoding chemokines (8-10, 12, 44).

IL-1β induces the expression of the iNOS gene, promoting marked accumulation of iNOS protein, a phenotype potentiated by the addition of IFN7 (13, 16, 17, 26). The active iNOS enzyme produces nitric oxide, a free radical signaling molecule that impacts numerous cellular functions (5, 28, 46). In pancreatic 3-cells, nitric oxide influences insulin secretion, DNA damage and repair, and overall cellular viability. In addition to controlling the abundance of iNOS, IL-1β also promotes increased production of a variety of chemokines (8, 44), which are soluble secreted factors that regulate immune cell recruitment and activation (25).

For example, CCL2 (a.k.a. MCP-1) is elevated in islets isolated from diabetic mice (8) and from human islets exposed to cytokines (21, 44). Transgenic mice with CCL2 production driven within pancreatic (3-cells display enhanced recruitment of immune cells into the pancreatic islets, although disease outcome differs depending on genetic background (35, 36). The chemokine CCL20 (a.k.a. LARC/MEP-3α) is also elevated within islets from mouse, rat, and humans during inflammation (11, 14, 44). CCL20 and CCL2 recruit distinct populations of leukocytes via the use of specific receptors. CCL2 signals through CCR2 (present on monocytes and macrophages), whereas CCL20 is a ligand for the CCR6 receptor (42).

Interestingly, CCL2, CCL20, and iNOS are all bona fide target genes controlled by the NF-kB family of transcription factors (8, 11, 13). NF-kB subunits include p65 (RelA), RelB, c-Rel, p50 (NF-kB 1), and p52 (NF-kB2). The NF-kB pathway is one of the major intracellular systems regulating inflammatory responses (2). Therefore, understanding the mechanisms underlying the IL-1β-mediated, NF-KB-regulated production of chemokines and other signaling molecules, such as nitric oxide, within pancreatic β-cells is essential for developing novel therapeutic strategies to prevent or reverse β-cell death and dysfunction. However, the precise mechanisms underlying the phenotypic changes in pancreatic β-cells in response to IL-1β and nitric oxide are not completely understood.

Toward this end, we have undertaken a systematic analysis of the timing of insulin secretion, nitric oxide accumulation, and chemokine production and release from pancreatic β-cells. We report herein that chemokine secretion increases, whereas insulin secretion decreases, in response to IL-1β. The NF-kB pathway is the central mediator of these outcomes. We further found that elevations in nitric oxide negatively regulate insulin secretion but have no effect on chemokine release. Moreover, the secretion of chemokines is not influenced by changes in glucose concentration but rather is controlled directly by NF-kB activity. We conclude that NF-kB is the central regulator of the reciprocal and coordinated changes in insulin and chemokine secretion in pancreatic β-cells during receipt of proinflammatory signals.

Materials and Methods:

Cell Culture, Islet Isolation, Adenoviral Vectors, and Reagents.

Culture of 832/13 and 833/15 rat insuUnoma cells and isolation of islets from Wistar and Zucker diabetic fatty (ZDF) rats were carried out as described previously (17, 18). All islet isolation protocols were approved by the Duke University and the University of Tennessee institutional animal care and use committees. All cell lines were confirmed to be free of mycoplasma contamination. The generation and use of adenoviruses encoding β-galactosidase (27) and IkBα^{sr} (32) have been reported. IL-β was purchased from Thermo Fisher Scientific (Waltham, Mass.), IFN-y was purchased from Shenandoah Biotechnology (Warwick, Pa.), Â-monomethyl-l-arginine (I-NMMA) was from Cayman Chemical (Ann Arbor, Mich.), and dimethyl malate was from Sigma Aldrich (St. Louis, Mo.). The spin trap N-methyl-d-glucamine dithiocarbamate (MGD) was from ENZO Life Sciences (Farmingdale, N.Y.).

Total RNA Isolation, cDNA Synthesis, and RT-PCR.

RNA isolation from cell lines and rat islets, cDNA synthesis, and analysis of real-time RT-PCR data has been described in detail previously (12). For all transcripts studied, the relative mRNA abundance was normalized to that of the housekeeping gene ribosomal S9. Primers used to detect transcript levels of ribosomal S9, iNOS, IL-1, and Nkx6.1 were designed using Primer3Plus software and are available upon request.

Cellular Fractionation and Immunoblot Analysis.

Cell lysis and extraction of cytoplasmic and nuclear protein fractions was performed using the NE-PER kit (Thermo Fisher Scientific) according to the manufacturer's directions. The protein concentration of the lysate was determined using the bicinchoninic acid assay (Thermo Fisher Scientific), using BSA as the standard. SDS-PAGE, transfer to polyvi-nylidene difluoride membranes, membrane blocking before antibody incubation, and downstream subsequent detection using a ChemiDoc imaging system (Bio-Rad) have been described previously (16). Antibodies detecting β-actin or tubulin were used as loading controls. Antibodies used were from the following sources: iNOS (Santa Cruz Biotechnology), (3-actin (Cell Signaling Technology), Ikα: (Santa Cruz Biotechnology), tubulin (Cell Signaling Technology), and Pdx-1 (Cell Signaling Technology). The antibody recognizing Nkx6.1 has been described (34). Immunoblot images were quantified using Image Lab software (Bio-Rad).

ELISA.

832/13 cells were grown in 12-well plates and treated as indicated. RPMI was supplemented with 0.3% BSA, and media were collected at the indicated time points. In addition, the cells were lysed using M-PER lysis reagent (Thermo Fisher Scientific) to quantify total intracellular protein content. CCL2 and CCL20 secretion into the media was measured by CCL2 Quantikine ELISA kit (R & D Systems, Minneapolis, Minn.) and CCL20 DuoSet ELISA kit (R & D Systems) according to the manufacturer's protocol. ELISA data were normalized to total protein to account for any differences in cell number between treatment groups.

Nitrite and MTS Assay.

Nitrite in the cell culture media was measured as an index of NO production using the Griess assay kit (Promega, Madison, Wis.), whereas cellular reduction of the 3-(4,5-dimethylthiazol-2-yl)-5-(3-carboxymethoxyphenyl)-2-(4-sulfophe-nyl)-2H-tetrazohum, inner salt (MTS) dye was determined using the MTS 1 solution kit (Promega) according to the manufacturer's protocol.

Electron Paramagnetic Resonance Spectroscopy.

The spin trap Af-methyl-d-glucamine dithiocarbamate-iron complex [(MGD)₂-Fe²⁺] was prepared fresh in each experiment by making stock solutions of 500 mM MGD and 100 mM Fe²⁺ (from FeS0₄.7 H₂0) in ultrapure water under anaerobic conditions. A final concentration of 25 mM MGD and 5 mM Fe²⁺ was introduced onto cells in culture using serum-free media with a 30-min incubation period. Supernatants were then collected for the measurements of the (MGD)₂-iron-NO complex. All electron paramagnetic resonance (EPR) measurements were carried out in a quartz flat cell at room temperature. Samples were transferred directly into the flat cell, which was then placed into the cavity of a Bruker EMX Plus spectroscope. Typical instrumental conditions were as follows: 20 mW of microwave power, 5.0-G modulation amplitude, 1×10⁵ gain, 0.163-s time constant, and 80-G scan range. Quantitation was carried out by measuring and comparing the first peak heights on the spectra.

Insulin Secretion and Tritiated Thymidine Incorporation Assays.

832/13 cells were grown in 12-well plates and treated as indicated. Glucose-stimulated insulin secretion (GSIS) assays were performed as described previously (29). Insulin secretion by cell lines into the media was measured using either the High Range Rat Insulin ELISA kit (Mercodia, Uppsala, Sweden) or by rat islets using the insulin radioimmunoassay (Siemens, Malvern, Pa.) according to the company directions. In addition, cells were lysed using M-PER lysis reagent (Thermo Fisher Scientific) to quantify total intracellular protein content via BCA assay (Thermo Fisher Scientific). Insulin secretion data were normalized to total protein to account for any differences in cell number between treatment groups. Incorporation of tritiated thymidine into DNA as a cellular index of proliferation was carried out as described (45).

Live Cell Calcium Imaging.

832/13 cells were plated into six-well plates containing poly-d-lysine-coated coverslips (Neuvitro, Vancouver, Wash.). At 50% confluence, cells were treated for 18 h with one of four experimental groups: 1 ng/ml IL-10 alone, 1 mM I-NMMA alone, IL-1β+ I-NMMA, or normal growth media. Immediately prior to imaging, cells were preloaded with Calcium Green 1 AM (CG, C3012; Life Technologies) by transferring coverslips to a solution of 10|×M CG in 20% Pluronic F127 in DMSO (Life Technologies) made up in carbogenated (95% 0₂-5% C0₂) normal Krebs. The recipe for normal Krebs solution was (in mM) 124 NaCl, 25 NaHC0₃, 2 glucose, 3 KC1, 2 CaCl₂, 1.5 NaH₂P0₄, and 1 MgS0₄-7H₂0, which corrected to pH 7.4 with HCl. After incubation in the CG solution for 30 min, coverslips were transferred to carbogenated normal Krebs at 29° C.

Coverslips were transferred to a recording chamber on a fixed stage Zeiss Axioskop2 microscope equipped with a Perkin Elmer Ultra-view/Yokogawa C10 spinning disk laser confocal illuminator. Cells were illuminated with the 488 nm laser; CG-calcium fluorescence signals were collected at 509 nm. Cells were visualized with a X40 water immersion objective. Images were collected with a Hammamatsu Ocra-ER charge-coupled device camera. The confocal system, including image collection, was controlled by Perkin-Elmer Improvision software.

Cells on coverslips were constantly perfused with normal Krebs solution at 29° C. at a rate of 3 ml/min. After 10 min, the perfusion solution was switched to one containing 12 mM glucose and proportionally less sodium chloride to maintain osmolality. The 12 mM glucose challenge was applied for 5 min, followed by a 5 min washout period. Images of CG-labeled cells were collected continuously during the challenge period at a rate of 20/min, with each frame taking 1 s. Images were collected as TIFF stacks and processed using Nikon Elements AR software. Individual cells in the field were designated as regions of interest (ROI), and their fluorescence signal over time was captured. Background fluorescence was subtracted from the fluorescence signal of individual ROIs. The relative changes in cytoplasmic calcium in the cells were expressed as changes in fluorescence [(AF/F) %], where F is the intensity of the baseline fluorescence signal before stimulation and AF is the difference between the peak fluorescence intensity and the baseline signal. Therefore, magnitude of response was normalized for each individual ROI.

Oxygen Consumption Assays.

Oxygen consumption rates (OCR) were measured using the XF²⁴ Extracellular Flux Analyzer (Seahorse Bioscience, Chicopee, Mass.). 832/13 cells were grown on cell culture plates for 2 days prior to OCR assays being run. Cells were treated with vehicle, 1 ng/ml IL-β, 1 mM I-NMMA, or both IL-1β (1 ng/ml) plus I-NMMA (1 mM) for 18 h prior to OCR being measured. The oxygen consumption rate was measured over a 2-h period. Data represent the calculated average area under the curve from six wells per treatment group over multiple independent experiments.

Mitochondrial/Nuclear DNA Ratio Analysis.

Genomic DNA was isolated using a DNeasy kit from Qiagen (Valencia, Calif.) per the manufacturer's protocol and quantified via quantitative PCR using iTaq Universal SYBR Green Supermix (Bio-Rad). Mitochondrial number was determined as the expression of genes encoded by mitochondrial DNA (COI and ATP6) relative to nuclear genes (NDUFA and SDHA) using the AAC_{T} method. This methodology has been described previously (24).

Electrophysiology.

Cell culture dishes were mounted on the stage of an Olympus K70 inverted microscope. A reference Ag/AgCl pellet served to ground the bath. Patch electrodes were pulled from thick-walled borosilicate glass with a filament (outer diameter 1.5 mm; inner diameter: 0.86 mm; Sutter Instruments, Novato, Calif.) using a Haming-Brown Micropipette Puller (Sutter Instruments). Electrode resistance values ranged from 4 to 7 Mfl. Ruptured patch, whole cell voltage clamp recordings were made using an Axopatch ID-patch clamp amplifier (Molecular Devices, Sunnyvale, Calif.). Data were recorded using pClamp 10.0 software (Molecular Devices). Unless otherwise indicated, all reagents were purchased from Sigma-Aldrich (St. Louis, Mo.). External solutions consisted of the following (in mM): 116.7 NaCl, 20.0 TEAC1, 3.0 CaCl₂, 0.4 MgCl₂, 4.0 glucose, and 10.0 HEPES. Voltage clamp experiments were performed in the ruptured patch configuration using the following internal solution (in mM): 100.00 CsAc, 10.0 CsCl, 2.0 MgCl₂, 0.1 CaCl₂, and 10.0 HEPES. Also included was an ATP regeneration system to preserve Ca²⁺ currents [3.0 ATP (dipotassium), 1.0 ATP (disodium), 20.0 phosphocreatine, 2.0 GTP, and 50 U/ml creatine phos-phokinase]. Cesium was used as a substitute for K⁺ to minimize currents through voltage-gated K⁺ channels. Tetrodotoxin (300 nM; Alomone Laboratories, Jerusalem, Israel) was included in the external solution to block voltage-gated Na⁺ currents. The liquid junction potential error for these solutions was estimated to be −13 mV (pClamp). Solutions were adjusted to pH 7.4 with NaOH for external solutions and with CsOH for internal solutions. A pressurized gravity flow perfusion system (1.5-2 mi/min) was used to deliver the external solutions (AutoMate Scientific, Berkeley, Calif.).

Statistical Analysis.

All statistical analyses were performed using GraphPad (La Jolla, Calif.) Prism 6.0 using one-way ANOVA, followed by Tukey's post hoc correction. For the live cell calcium imaging, mean magnitude of responses for each experimental condition were determined and analyzed using one-way ANOVA with Dunnett's multiple comparison pos-test analysis. Significance values are given in the figure legends.

Results:

IL-1β Reciprocally Regulates Chemokine and Insulin Secretion.

For decades, IL-1β has been associated with inhibition of GSIS (43). Indeed, we observed a steady decrease in GSIS over a time course of exposure to 1 ng/ml IL-1β, with a 40 and 59% decrease in GSIS by 6 and 12 h, respectively (FIG. 5). In contrast to GSIS, the expression and secretion of the chemokine CCL2 are markedly elevated in response to IL-1β (FIG. 5 and data not shown). These data demonstrate the reciprocal regulation of chemokine and insulin secretion by IL-1β (FIG. 5).

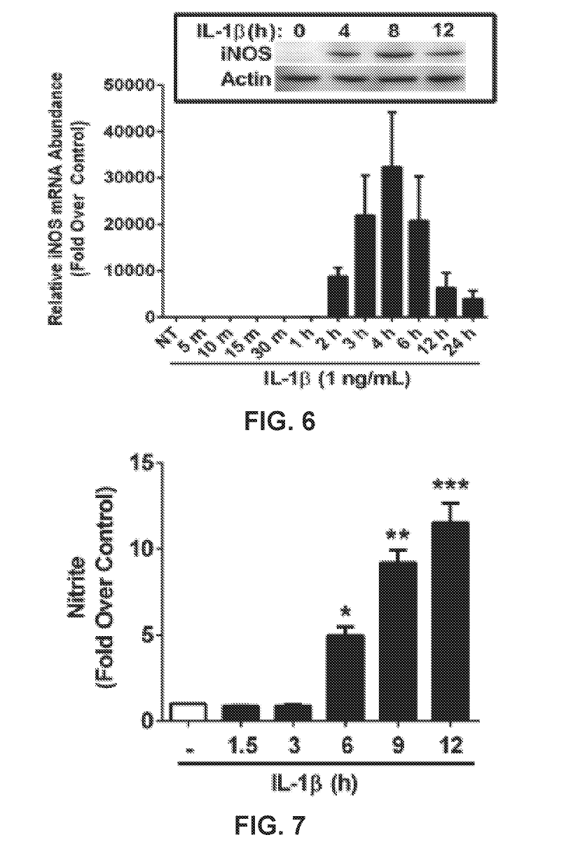

Because elevations in nitric oxide have been linked with decreases in GSIS (19, 20), we investigated the timing of iNOS mRNA accumulation in response to IL-1β. iNOS expression is undetectable at both the transcript and protein level in the absence of IL-1β but is rapidly induced upon cellular exposure to IL-1β (FIG. 6). The marked elevation in transcript abundance FIG. 6) is congruent with the increase in iNOS protein (FIG. 6, inset) and nitrite accumulation (FIG. 7), an index of nitric oxide production. Thus, the accumulation of nitrite correlates with the decrease in GSIS (compare FIGS. 5 and 7). We conclude that IL-ip reciprocally regulates insulin and chemokine secretion in pancreatic β-cells.

NF-kB is Required for Cytokine-Mediated Decreases in Insulin Secretion and Cellular Production of Nitric Oxide.

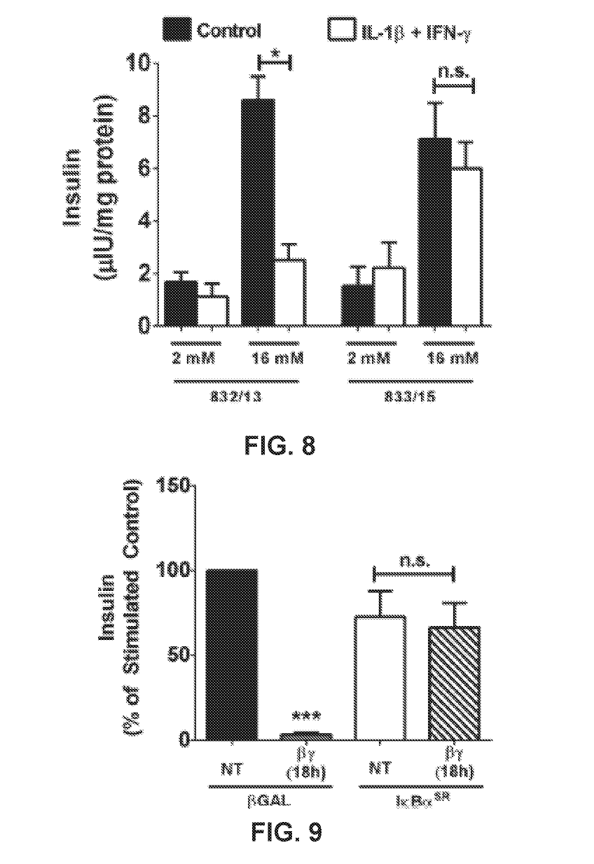

The NF-kB pathway is strongly activated by IL-1β in pancreatic β-cells (6, 16), and the proinflammatory cytokines IL-1β and IFN-y impair p-cell function and viability through activation of specific signaling pathways such as NF-kB and STAT1 (16, 17, 26). The clonal β-cell line 833/15 is resistant to losses in viability upon exposure to IL-1β and WNy (17, 47). Using clonal β-cell lines, which are either sensitive (832/13) or resistant (833/15) to killing by IL-1β+IFN-y, we tested whether or not insulin secretion was preserved in the cytokine-resistant 833/15 cells. Under normal conditions (i.e., no cytokines present), GSIS was similar between 832/13 and 833/15 cells (FIG. 8, black bars). However, when cultured overnight in the presence of IL-1β and IFNy, only 833/15 cells retained the ability to secrete insulin in response to a glucose stimulus (FIG. 8, open bars).

An inherent defect in NF-kB-mediated gene activation in the 833/15 cell line was reported (15), which we interpreted as the reason for their protection against cytokine-mediated impairments in GSIS. To directly test this possibility, we used adenoviral delivery of a mutated form of IkBα that contains S32A/S36A substitutions, termed the IkBα: super-repressor (IkBα<^{sr}). The IkBα^{sr} retains the p65 subunit of NF-kB in the cytosol and thus decreases NF-kB transcriptional activity (11, 16). Culturing rat islets in IL-1β and IFN-y overnight completely suppressed GSIS; this loss in function was reversed in islets transduced with the IkBα^{sr}, but not in islets receiving a control virus expressing (i-galactosidase (FIG. 9).

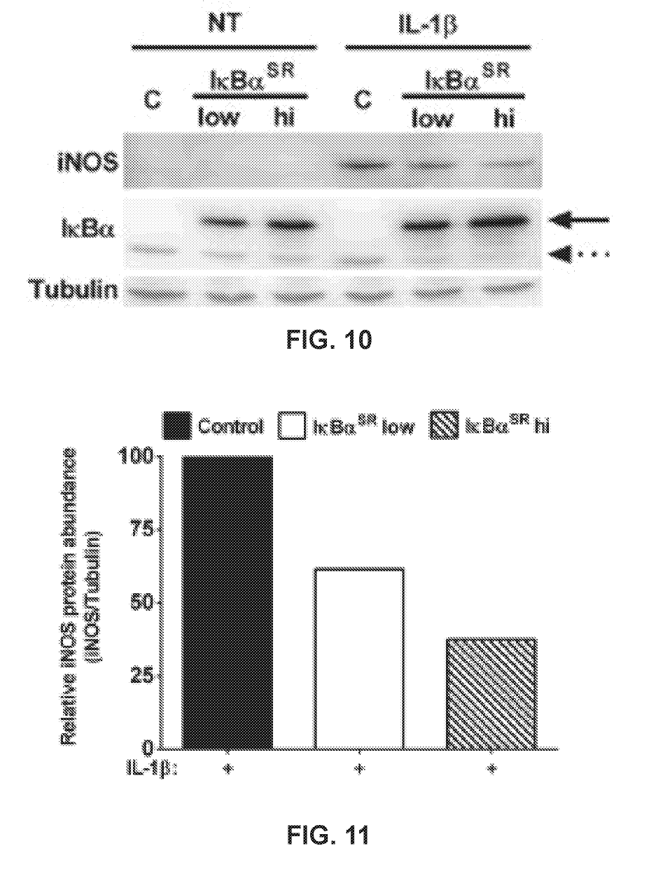

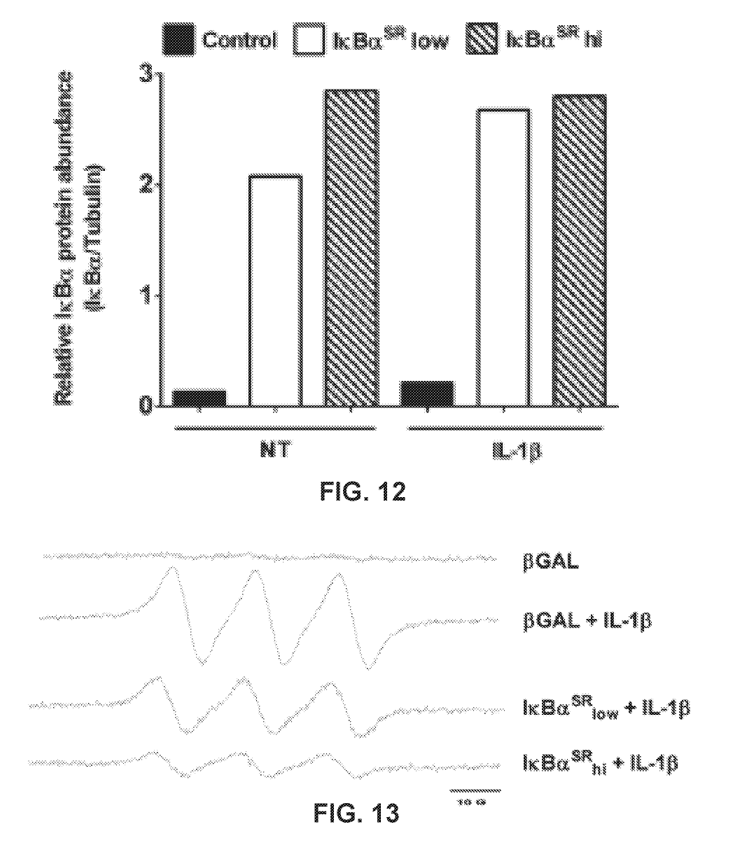

To investigate the contribution of NF-kB to nitric oxide production after cellular exposure to IL-1β, we first analyzed iNOS protein abundance in the presence and absence of the IkBα:^{sr}. Again, iNOS protein is undetectable in the absence of a cytokine stimulus but accumulates upon cellular exposure to IL-1β (FIG. 10). Inhibiting NF-kB transcriptional activity, using adenoviral transduction of 832/13 cells with IkBα:^{sr}, decreases iNOS protein abundance by 73% at the highest viral dose (FIG. 10; quantified in FIGS. 11 and 12).

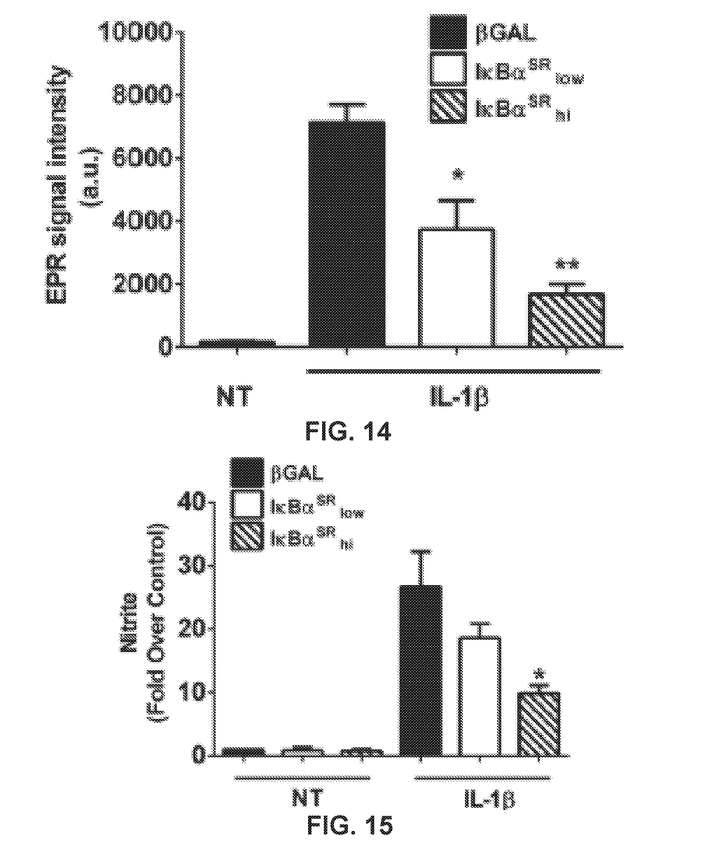

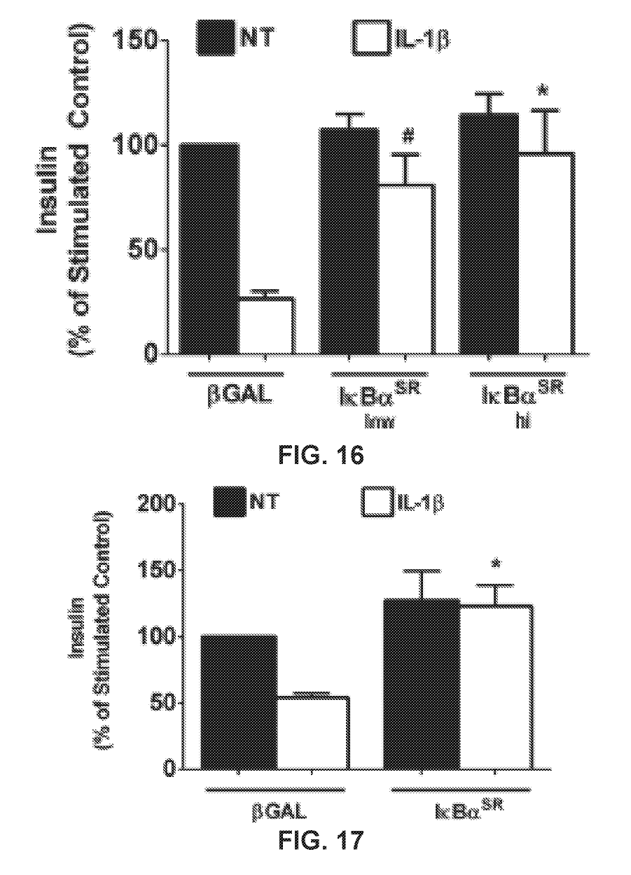

Using EPR spectroscopy in conjunction with the spin trap (MGD)2Fe²⁺, we observed robust production of nitric oxide in response to IL-1β (spectra shown in FIG. 13). This technique specifically measures nitric oxide radicals. Reducing iNOS expression with the IkBα:^{sr} diminished nitric oxide production in a manner consistent with the decrease in iNOS protein (FIGS. 10 and 13). Quantification of the signal intensity from multiple EPR experiments is shown in FIG. 14. The Griess assay, which measures total nitrite accrual in the media from both organic and inorganic sources, also revealed decreased nitrite production when iNOS protein levels were reduced by blocking NF-kB activity (FIG. 15). This diminution in nitric oxide levels correlated directly with the complete recovery of IL-1β-mediated impairments in GSIS in 832/13 clonal β-cells cultured for 6 (FIG. 16) and 18 h (FIG. 17) with IL-1β. We interpreted these data to indicate that transcriptional activation of the iNOS gene by NF-kB controls iNOS enzyme accumulation and subsequent nitric oxide production, which serves as a negative regulator of insulin secretion.

NF-kB is Required for IL-β-Mediated Decreases in Cellular Proliferation.

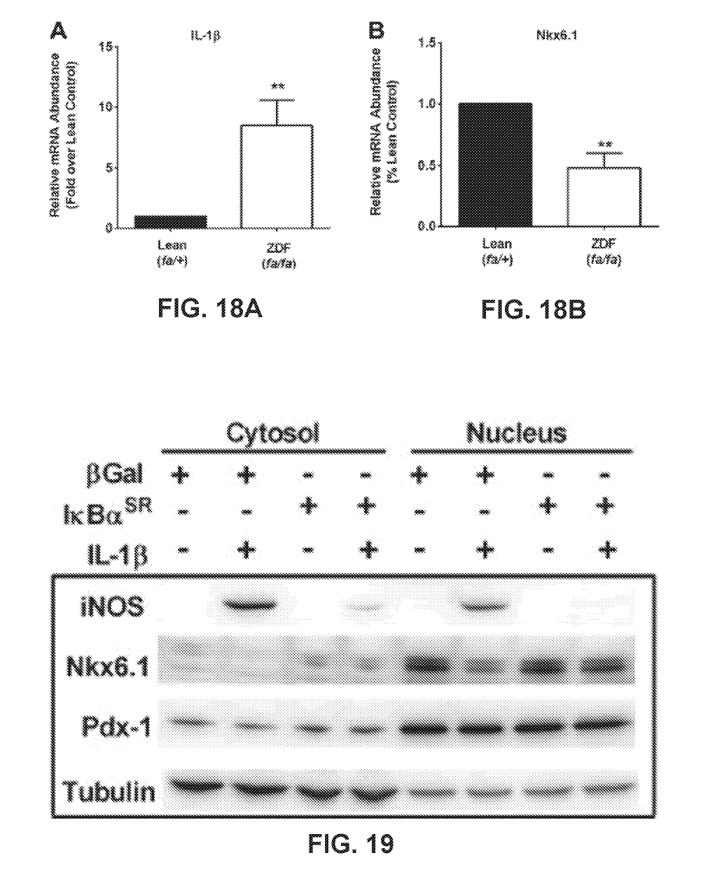

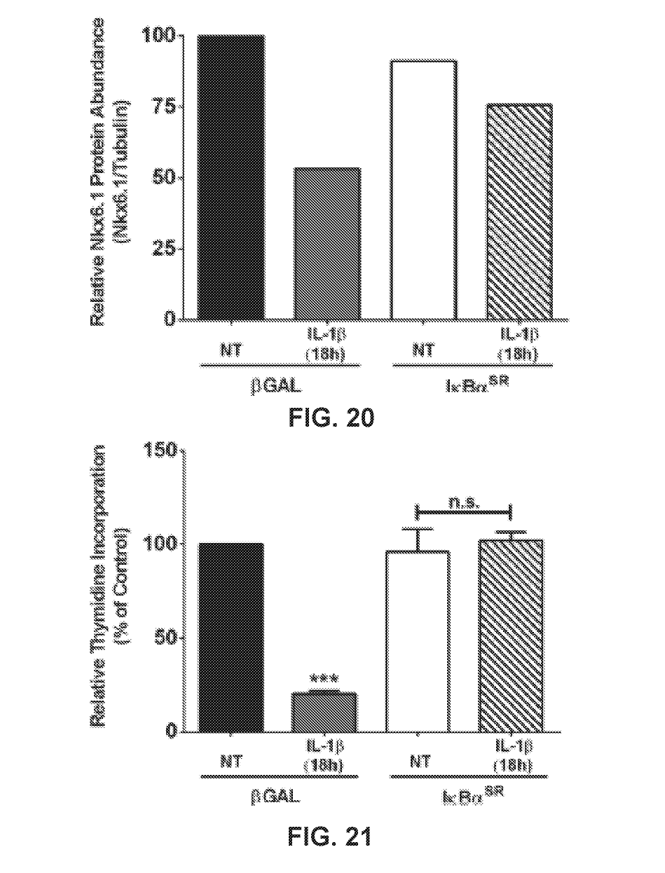

A reduction in islet β-cell mass and function is a hallmark of both T1DM and T2DM. The Zucker diabetic fatty (ZDF) rat is a model of obesity and diabetes resulting from reduced functional β-cell mass (4). Using isolated islets from 8-wk-old ZDF rats, we found that IL-1β expression was elevated 8.6-fold (FIG. 18A). Nkx6.1 expression was reduced by 52% in islets isolated from ZDF rats relative to their lean littermate controls (FIG. 18B). Because the transcription factor Nkx6.1 regulates β-cell proliferation (45), we next examined its abundance under conditions of IL-1β exposure in the presence or absence of the IkBα:^{sr}. 832/13 cells exposed to IL-1β for 4 h displayed robust iNOS abundance, which was blocked by the IkBα^{sr} (FIG. 3C). Nkx6.1 was largely nuclear, and its abundance was decreased by 47% after IL-1β exposure (FIGS. 19-20). Moreover, the ability to incorporate thymidine into DNA, an index of proliferation, was reduced markedly in 832/13 (3-cells exposed to IL-1β for 18 h but was restored in cells expressing the IkBα^{SR} (FIG. 20). Thus, blocking NF-kB activity with the IkBα^{sr} restored Nkx6.1 abundance (FIG. 19), which was consistent with the recovery in cellular thymidine incorporation (FIG. 21). The amount of Pdx-1 protein was unchanged under these conditions. These observations are consistent with IL-1β-mediated decreases in proliferation being controlled by NF-kB activation.

IL-1β Generates Elevations in Nitric Oxide Production and Decreases in Oxygen Consumption.

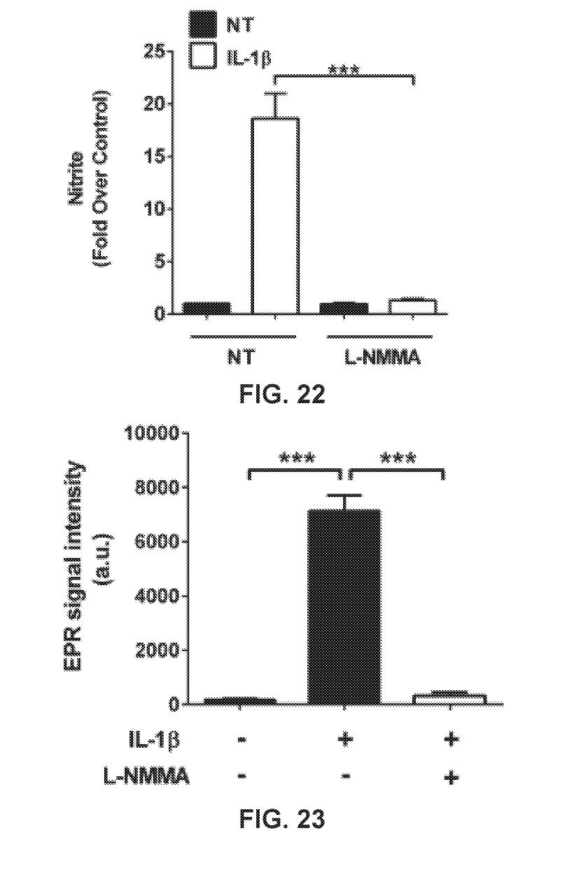

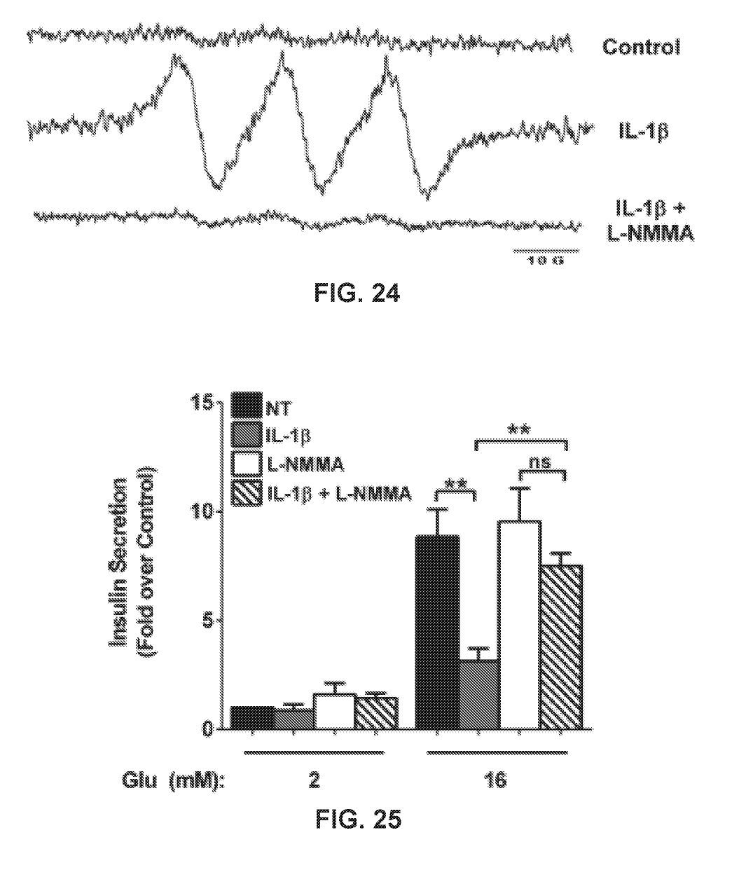

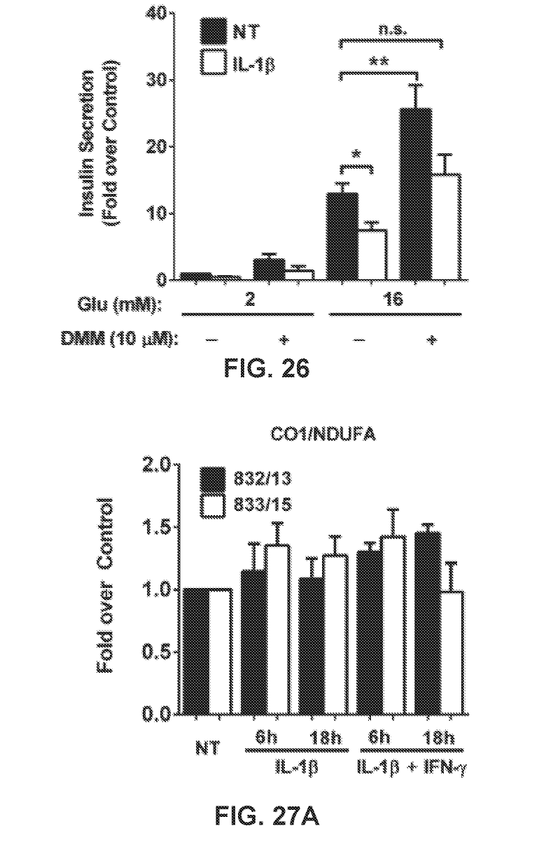

LL-1β promotes robust accumulation of the iNOS protein (FIGS. 5-9 and Ref. 26) and nitrite accumulation, which impairs insulin secretion; this effect can be prevented by blocking NF-kB transcriptional activity (FIGS. 5-17). Therefore, we used I-NMMA, an arginine analog that inhibits all known nitric oxide synthase isoforms (39), and found that the increase in total nitrite production by cellular exposure to IL-1β was completely inhibited (FIG. 22). The EPR (MGD)₂ Fe²⁺ spin trap methodology also revealed a lack of IL-Iβ-mediated elevations in nitric oxide formation when I-NMMA was present (FIG. 23); representative spectra shown in FIG. 24). Therefore, we next addressed whether I-NMMA prevented the suppression of insulin secretion upon exposure to IL-1β. We observed an almost complete recovery of insulin secretion in the presence of IL-1β+ I-NMMA (FIG. 25). In addition, insulin secretion could be recovered by dimethyl malate (FIG. 26), a malate analog, indicating that mitochondrial metabolism was still active in the presence of IL-1β.

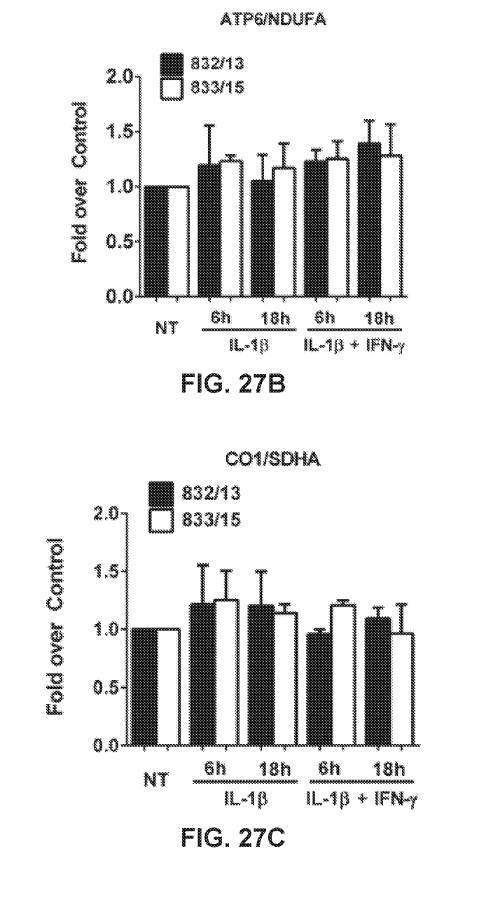

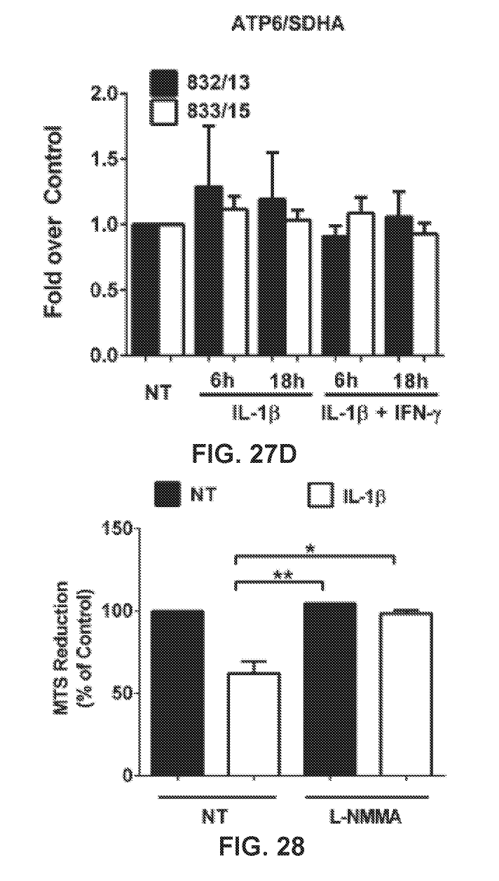

To address whether mitochondrial number or mitochondrial function is altered by IL-β, we first measured the mitochondrial/nuclear DNA ratios, which have been used to track changes in mitochondrial biogenesis (24). We did not detect any alterations in mitochondrial/nuclear DNA ratios (FIGS. 27A-27D), which we interpreted as evidence that mitochondrial number was not being altered in response to either IL-1β or the combination of IL-1β+IFNy.

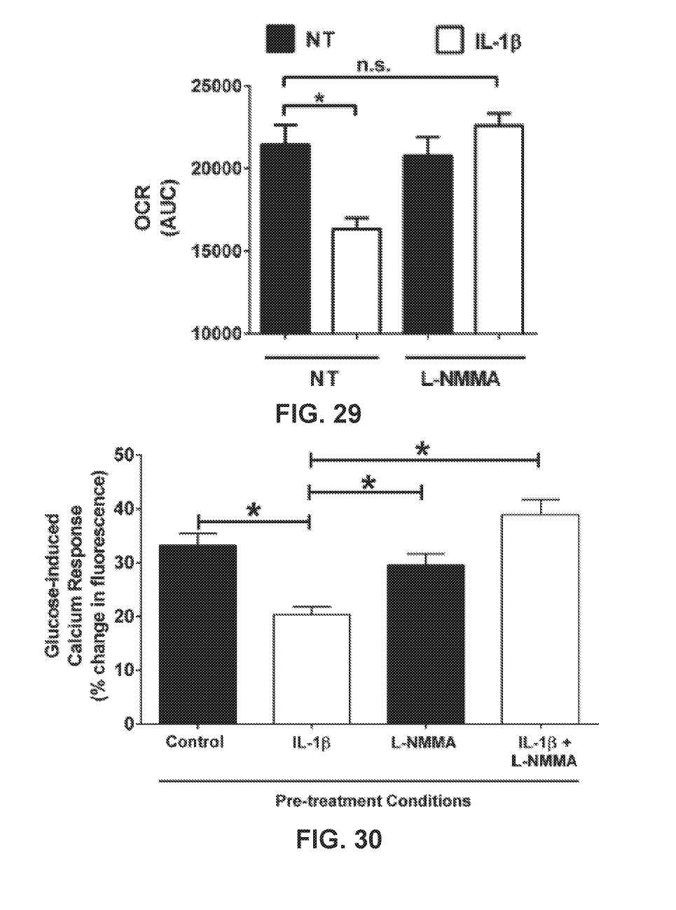

Therefore, we next investigated whether mitochondrial function was reduced. We found a 38% decrease in reduction of the MTS dye after IL-1β exposure (FIG. 28). This decrease was fully recovered in the presence of the NOS inhibitor I-NMMA (FIG. 28). In addition, a 25% decrease in cellular oxygen consumption rate was identified, which was also prevented by the addition of 1 mM I-NMMA (FIG. 29). We conclude that enhanced cellular abundance of nitric oxide diminished mitochondrial activity but did not alter total mitochondrial number.

Nitric Oxide Mediates the IL-1β-Induced Impairment in Glucose-Stimulated Calcium Elevations.

Nitric oxide suppresses insulin secretion in mouse, rat, and human islets (5). Using 832/13 rat clonal β-cells, which display similar impairments in insulin secretion, as observed in isolated islets, we examined changes in cytoplasmic calcium using the reporter dye calcium green in conjunction with confocal microscopy. In control pretreated cells, −97% of the cells responded to the glucose challenge with an averaged response magnitude of 33% change in fluorescence levels. In cells pretreated with DL-ip alone, only 61% of the cells responded to the glucose challenge at an average response magnitude of 20%. However, the pretreat-ment of cells with I-NMMA plus IL-1β rescued both the number of responsive cells (89%) and the magnitude of the response (30%) of these cells to the glucose challenge. Cells pretreated with I-NMMA alone differed neither in the number of responders nor in the magnitude of the responses to the glucose challenge relative to control pretreatment. Thus, the responses of cells pretreated with IL-1β alone were reduced by 39% relative to the responses of the control group (FIG. 30). This reduced change in intracellular calcium observed with IL-1β was completely prevented by 1 mM I-NMMA (FIG. 30), indicating a requirement for nitric oxide production to alter the glucose-induced increase in cytoplasmic calcium levels.

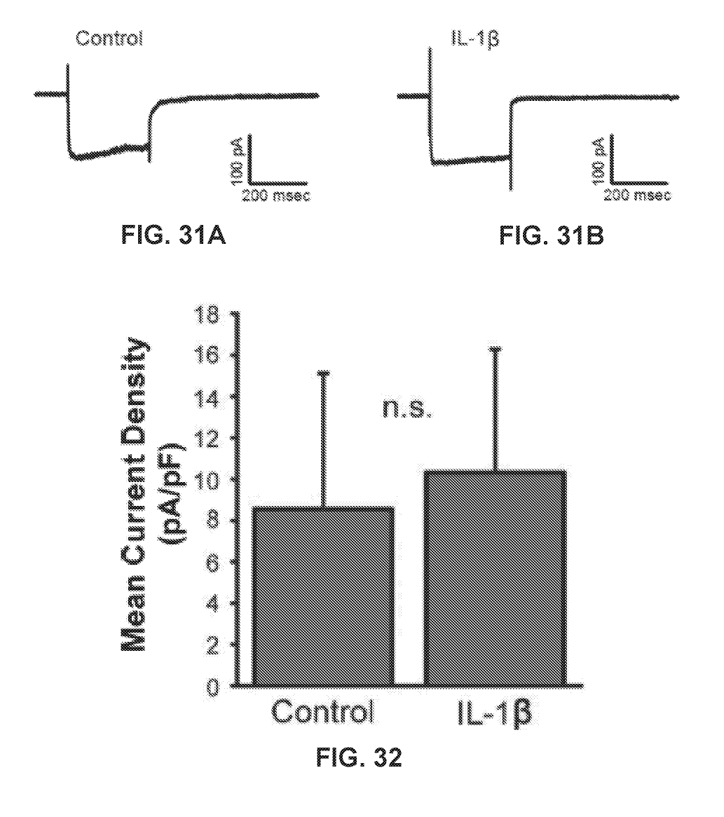

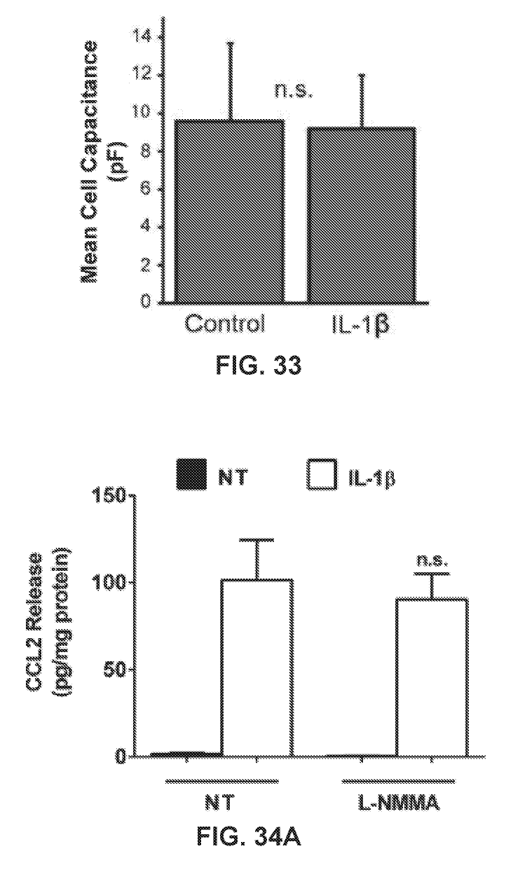

To test for a decrease in the activity of voltage-gated Ca²⁺ channels, whole cell voltage clamp recordings were made from individual 832/13 clonal β-cells (FIGS. 31A-31B). Voltage-gated Ca²⁺ current density was not significantly different in control or IL-1β-treated cells (FIG. 32, and IL-1β pretreatment also had no effect on cell size as indicated by whole cell capacitance measurements (FIG. 33). We conclude that nitric oxide accumulation most likely decreases calcium release from internal stores and not by reducing the activity of voltage-gated calcium channels.

NF-kB, but not Glucose or Nitric Oxide Concentration, Controls Production of the Chemokines CCL2 and CCL20.

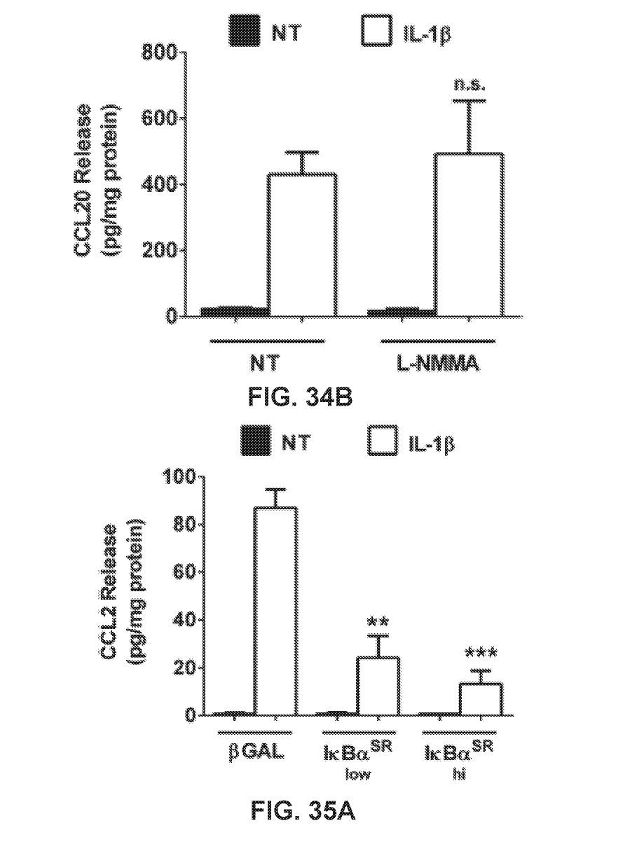

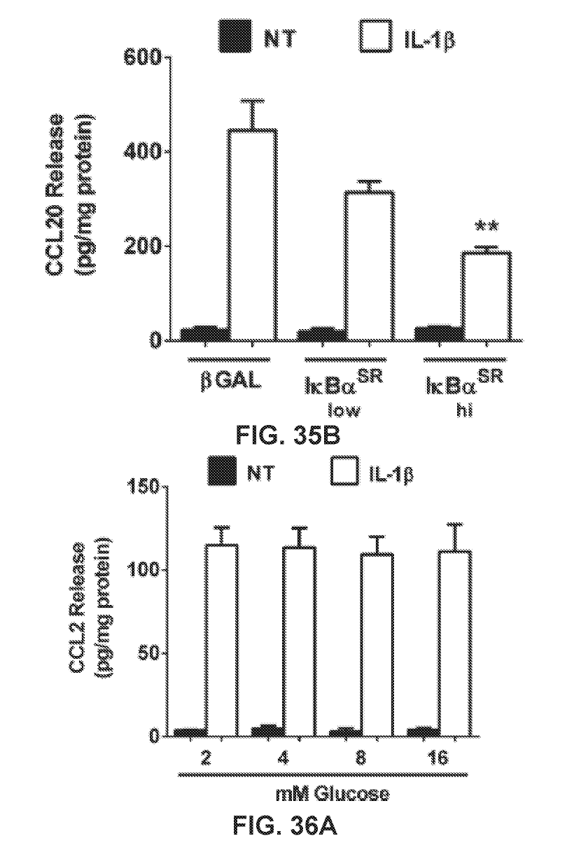

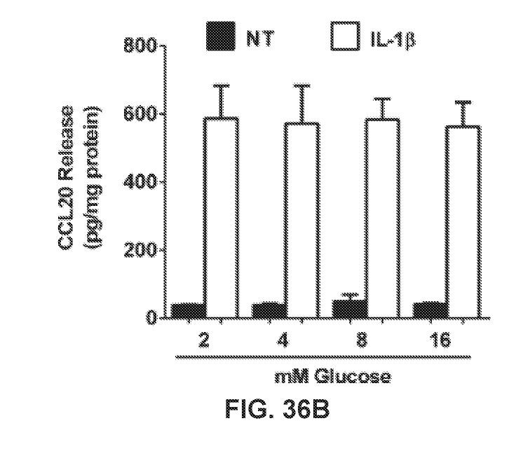

Because nitric oxide is a signaling molecule whose elevated abundance reduces insulin secretion, we next investigated whether chemokine secretion, a process also driven by IL-Iβ, was altered by changes in either glucose or nitric oxide concentration. IL-1β strongly induced the synthesis and secretion of CCL2 in pancreatic β-cells, which are not altered by the addition of 1 mM I-NMMA (FIG. 34A). Similarly, CCL20 secretion is not impacted by suppressing nitric oxide production using I-NMMA (FIG. 34B). By contrast, inhibiting NF-kB transcriptional activity via adenoviral delivery of the IkBa^{SR} dose dependently decreases the expression of the CCL2 gene (Ref. 10 and data not shown) and release of CCL2 protein (FIG. 35A). Similar results were observed for CCL20 release (FIG. 35B). Finally, we also investigated whether chemokine secretion was dependent on the glucose concentration. Although glucose concentration is the major determinant controlling insulin secretion (37), the IL-1β-induced secretion of both CCL2 and CCL20 is not different over a range of glucose concentrations (FIGS. 36A-36B).

Discussion:

A diminution in the function and mass of pancreatic islet β-cells is a hallmark of both major forms of diabetes (1, 7). Although extended exposure of islet p-cells to proinflammatory cytokines decreases viability, less is known about the early changes that occur in response to the initial transcriptional reprogramming events. Therefore, we undertook the present study to investigate the acute functional responses of β-cells to the proinflammatory cytokine IL-1β. Several novel observations emerged. (7) Chemokine secretion occurred rapidly (within 3-6 h) after cellular exposure to IL-1β; 2) insulin secretion began to decrease by 6 h, concomitant with elevations in nitric oxide production and reduction in the abundance of Nkx6.1. 3) the decline in proliferation after exposure to IL-1β was consistent with decreases in Nkx6.1 abundance; 4) all of these processes were reversed by blocking NF-kB transcriptional activity; and 5) chemokine release was not dependent on glucose concentration or nitric oxide accumulation, whereas elevations in cellular nitric oxide levels negatively regulated insulin secretion.

The 5.5-fold increase in nitrite accrual (Fig. IS) at 6 h is connected with a 36% decrease in GSIS, whereas a 13-fold increase in nitrite is associated with a 54% diminution in GSIS (FIG. 6). Because measurements of total nitrite production by the Griess method account for both organic and inorganic nitrite-containing compounds, we used the MGD spin trap coupled with EPR spectroscopy to unequivocally measure nitric oxide production. Accrual of nitric oxide was inversely correlated with insulin secretion, implicating this free radical signaling molecule as a negative regulator of insulin release in β-cells. However, the mitochondria are still functional in β-cells exposed to IL-1β, as evidenced by enhanced insulin secretion in the presence of dimethyl malate (DMM; a malate analog). Because mitochondrial substrate metabolism is tightly linked with insulin secretion (37), the ability of DMM to rescue insulin secretion is consistent with nitric oxide functioning as an intracellular signaling intermediary to decrease insulin secretion after exposure to IL-1β.

DMM also improves insulin secretion in islets isolated from ZDF rats and clonal p-cells cultured in lipids (4). We have shown herein that ZDF islets display elevated levels of IL-1β mRNA relative to lean control islets (FIGS. 18A-21). This finding is consistent with IL-1β serving as a common link to alterations in islet function and mass in both T1DM and T2DM. Furthermore, if nitric oxide is removed from cells within the first 24 h of IL-1β exposure, mitochondrial metabolism and insulin secretion are restored (5). In this study, we found that voltage-gated calcium channel activity is not different between control and IL-1β-exposed cells, but the ability to increase intracellular calcium in response to a glucose challenge is diminished (FIGS. 30-33). Thus, nitric oxide accumulation most likely prevents the release of calcium from internal storage depots, which is consistent with reduced insulin release observed in our studies and complementary to findings from previous studies (38). Collectively, we interpret these data to indicate that the mitochondria are responsive to both nitric oxide and metabolic signals and that an initial increase in intracellular nitric oxide production acts as a rheostat to rapidly and reversibly diminish insulin secretion in response to a specific inflammatory signal (e.g., IL-1β) via multiple mechanisms.

In contrast to insulin secretion, the synthesis and release of chemokines in response to IL-1β is completely independent of either the glucose concentration or nitric oxide accumulation. We discovered that the major control point for both IL-1β-mediated reduction in insulin secretion and enhancements in chemokine production is NF-kB activation. Indeed, several chemokines are primary response genes in pancreatic p-cells exposed to IL-1β (Refs. 8 and 12 and data not shown), which may be explained at least in part by the exquisite sensitivity of the pancreatic β-cell to IL-1 receptor (IL-1R) activation. The marked sensitivity of β-cells to IL-1β is due largely to the high level of IL-1R expression (3). Thus, at least one of the major consequences of islet p-cell signaling via IL-1R ligands (e.g., IL-1β) is the rapid and reciprocal regulation of chemokine and insulin secretion. Consequently, the islet β-cell may play a much more important role in regulating immune system function than has been recognized previously. Along these lines, insulin has documented anti-inflammatory properties (30, 31). Therefore, p-cell insulin release may have a greater impact on innate and adaptive immunity than realized previously, which would fit with a need to coordinately downregulate insulin secretion during periods of inflammation. Because chemokines control both immune cell recruitment as well as immune cell activity, changes in the islet p-cell ratio of insulin to chemokine secretion could have important consequences for both local and systemic inflammatory responses as well as for metabolic homeostasis.

The progression to both T1DM and T2DM requires immune cell-associated alterations in islet β-cell mass and function. With many discrete chemokines made in response to IL-1β and IFN7 in pancreatic β-cells (8, 44), the p-cell influence on tissue leukocytosis and overall pancreatic inflammatory responses may be underappreciated. It also appears that NF-kB is the major regulatory factor controlling chemokine production in response to IL-1β (9, 10, 12), whereas STAT1 is the predominant transcription factor mediating the increased chemokine production by IFNy (9, 12). Furthermore, NF-kB is the key determinant of the chemokine/insuhn secretion ratio, suggesting that modulation of NF-kB would be beneficial for improving islet function by suppressing inflammatory responses. The results of a variety of in vitro and in vivo studies support this rationale (21-23, 33, 40, 41), and a recent computational model predicted elevated IL-1β signaling as necessary and sufficient to promote progression to T2DM (48).

In summary, we have shown herein that IL-1β signaling via NF-kB controls both insulin and chemokine secretion and also mediates decreases in p-cell proliferation. Thus, the transcriptional reprogramming of β-cells, such as that which occurs after exposure to IL-1β, is a major biochemical and molecular process controlling both islet p-cell insulin secretion and pancreatic leukocyte infiltration via chemokine release. Understanding the molecular mechanisms underlying these signal-induced genetic events should provide novel insights into therapeutic possibilities to prevent loss in β-cell function and mass.

### REFERENCES

1. Ashcroft F M, Rorsman P. Diabetes mellitus and the beta cell: the last ten years. Cell 148: 1160-1171, 2012.
2. Baker R G, Hayden M S, Ghosh S. N F-kappaB, inflammation, and metabolic disease. Cell Metab 13: 11-22, 2011.
3. Boni-Schnetzler M, Boiler S, Debray S, Bouzakri K, Meier D T, Prazak R, Kerr-Conte J, Pattou F, Ehses J A, Schuit F C, Donath M Y. Free fatty acids induce a proinflammatory response in islets via the abundantly expressed interleukin-1 receptor I. Endocrinology 150: 5218-5229, 2009.
4. Boucher A, Lu D, Burgess S C, Telcmaque-Potts S, Jensen M V, Mulder H, Wang M V, Unger R H, Sherry A D, Newgard C B. Biochemical mechanism of lipid-induced impairment of glucose-stimulated insulin secretion and reversal with a malate analogue. J Biol Chem 279: 27263-27271, 2004.
5. Broniowska K A, Oleson B J, Corbett J A. (5-Cell responses to nitric oxide. Vitam Horm 95: 299-322, 2014.
6. Burke S J, Collier J J. The gene encoding cyclooxygenase-2 is regulated by JJL-lbeta and prostaglandins in 832/13 rat insulinoma cells. Cell Immunol 271: 379-384, 2011.
7. Burke S J, Collier J J. Insulitis and diabetes: a perspective on islet inflammation. Immun Res 10: e002, 2014.
8. Burke S J, Collier J J. Transcriptional regulation of chemokine genes: a link to pancreatic islet inflammation? Biomolecules 5: 1020-1034, 2015.
9. Burke S J, Goff M R, Lu D, Proud D, Karlstad M D, Collier J J. Synergistic expression of the CXCL10 gene in response to IL-ip and IFN-7 involves NF-kB, phosphorylation of STAT1 at Tyr701, and acet-ylation of histones H3 and H4. J Immunol 191: 323-336, 2013.
10. Burke S J, Goff M R, Updegraff B L, Lu D, Brown P L, Minkin S C Jr, Biggerstaff J P, Zhao L, Karlstad M D, Collier J J Regulation of the CCL2 gene in pancreatic p-cells by IL-ip and glucocorticoids: role of MKP-1. PLoS One 7: e46986, 2012.
11. Burke S J, Karlstad M D, Regal K M, Sparer T E, Lu D, Elks C M, Grant R W, Stephens J M, Burk D H, Collier J J. CCL20 is elevated during obesity and differentially regulated by NF-kappaB subunits in pancreatic beta-cells. Biochim Biophys Acta 1849: 637-652, 2015.
12. Burke S J, Lu D, Sparer T E, Masi T, Goff M R, Karlstad M D, Collier J J. NF-kB and STAT1 control CXCL1 and CXCL2 gene transcription. Am J Physiol Endocrinol Metab 306: E131-E149, 2014.
13. Burke S J, Updegraff B L, Bellich R M, Goff M R, Lu D, Minkin S C Jr, Karlstad M D, Collier J J Regulation of iNOS gene transcription by JJL-1 p and IFN-7 requires a coactivator exchange mechanism. Mol Endocrinol 27: 1724-1742, 2013.
14. Cardozo A K, Proost P, Gysemans C, Chen M C, Mathieu C, Eizirik D L. IL-Ibeta and JFN-gamma induce the expression of diverse chemokines and EL-15 in human and rat pancreatic islet cells, and in islets from pre-diabetic NOD mice. Diabetologia 46: 255-266, 2003.
15. Chen G, Hohmeier H E, Gasa R, Tran W, Newgard C B. Selection of insuUnoma cell lines with resistance to interleukin-lbeta- and gamma-interferon-induced cytotoxicity. Diabetes 49: 562-570, 2000.
16. Collier J J, Burke S J, Eisenhauer M E, Lu D, Sapp R C, Frydman C J, Campagna S R. Pancreatic p-cell death in response to pro-inflammatory cytokines is distinct from genuine apoptosis. PLoS One 6: e22485, 2011.
17. Collier J J, Fueger P T, Hohmeier H E, Newgard C B. Pro- and antiapo-ptotic proteins regulate apoptosis but do not protect against cytokine-mediated cytotoxicity in rat islets and beta-cell lines. Diabetes 55: 1398-1406, 2006.
18. Collier J J, White S M, Dick G M, Scott D K. Phosphatidylinositol 3-ki-nase inhibitors reveal a unique mechanism of enhancing insulin secretion in 832/13 rat insulinoma cells. Biochem Biophys Res Commun 324: 1018-1023, 2004.
19. Corbett J A, Sweetland M A, Wang J L, Lancaster J R Jr, McDaniel M L.
Nitric oxide mediates cytokine-induced inhibition of insulin secretion by human islets of Langerhans. Proc Natl Acad Sci USA 90: 1731-1735, 1993.
20. Corbett J A, Wang J L, Sweetland M A, Lancaster J R Jr, McDaniel M L. Interleukin 1 beta induces the formation of nitric oxide by beta-cells purified from rodent islets of Langerhans. Evidence for the beta-cell as a source and site of action of nitric oxide. J Clin Invest 90: 2384-2391, 1992.
21. Cowley M J, Weinberg A, Zammit N W, Walters S N, Hawthorne W J, Loudovaris T, Thomas H, Kay T, Gunton J E, Alexander S I, Kaplan W, Chapman J, O'Connell P J, Grey S T. Human islets express a marked proinflammatory molecular signature prior to transplantation. Cell Transplant 21: 2063-2078, 2012.
22. Ding X, Wang X, Xue W, Tian X, Li Y, Jiao F, Feng X, Zheng J. Blockade of the nuclear factor kappa B pathway prolonged islet allograft survival. Artif Organs 36: E21-E27, 2012.
23. Eldor R, Yeffet A, Baum K, Doviner V, Amar D, Ben-Neriah Y, Christofori G, Peled A, Carel J C, Boitard C, Klein T, Serup P, Eizirik D L, Meiloul D. Conditional and specific NF-kappaB blockade protects pancreatic beta cells from diabetogenic agents. Proc Natl Acad Sci USA 103: 5072-5077, 2006.
24. Ellis J M, Mentock S M, Depetrillo M A, Koves T R, Sen S, Watkins S M, Muoio D M, Cline G W, Taegtmeyer H, Shulman G I, Willis M S, Coleman R A. Mouse cardiac acyl coenzyme a synthetase 1 deficiency impairs Fatty Acid oxidation and induces cardiac hypertrophy. Mol Cell Biol 31: 1252-1262, 2011.
25. Gerard C, Rollins B J. Chemokines and disease. Nat Immunol 2: 108-115, 2001.
26. Heitmeier M R, Scarim A L, Corbett J A. Interferon-gamma increases the sensitivity of islets of Langerhans for inducible nitric-oxide synthase expression induced by interleukin 1. J Biol Chem 272: 13697-13704, 1997.
27. Herz J, Gerard R D. Adenovirus-mediated transfer of low density lipoprotein receptor gene acutely accelerates cholesterol clearance in normal mice. Proc Natl Acad Sci USA 90: 2812-2816, 1993.
28. Hill B G, Dranka B P, Bailey S M, Lancaster J R Jr, Darley-Usmar V M. What part of NO don't you understand? Some answers to the cardinal questions in nitric oxide biology. J Biol Chem 285: 19699-19704, 2010.
29. Hohmeier H E, Mulder H, Chen G, Henkel-Rieger R, Prentki M, Newgard C B. Isolation of JNS-I-derived cell lines with robust ATP-sensitive K+ channel-dependent and -independent glucose-stimulated insulin secretion. Diabetes 49: 424-430, 2000.
30. Jeschke M G, Klein D, Bolder U, Einspanier R. Insulin attenuates the systemic inflammatory response in endotoxemic rats. Endocrinology 145: 4084-4093, 2004.
31. Jeschke M G, Klein D, Herndon D N. Insulin treatment improves the systemic inflammatory reaction to severe trauma. Ann Surg 239: 553-560, 2004.
32. Jobin C, Panja A, Hellerbrand C, Iimuro Y, Didonato J, Brenner D A, Sartor R B. Inhibition of proinflammatory molecule production by adenovirus-mediated expression of a nuclear factor kappaB super-repressor in human intestinal epithelial cells. J Immunol 160: 410̂118, 1998.
33. Kanak M A, Takita M, Itoh T, SoRelle J A, Murali S, Kunnathodi F, Shahbazov R, Lawrence M C, Levy M F, Naziruddin B. Alleviation of instant blood-mediated inflammatory reaction in autologous conditions through treatment of human islets with NF-kB inhibitors. Transplantation 98: 578-584, 2014.
34. Klinck R, Serup P, Madsen O D, Jorgensen M C. Specificity of four monoclonal anti-NKx6-I antibodies./Histochem Cytochem 56: 415̂124, 2008.
35. Kriegel M A, Rathinam C, Flavell R A. Pancreatic islet expression of chemokine CCL2 suppresses autoimmune diabetes via tolerogenic CDIIc+ CDIIb+ dendritic cells. Proc Natl Acad Sci USA 109: 3457-3462, 2012.
36. Martin A P, Rankin S, Pitehford S, Charo I F, Furtado G C, Lira S A.
37. Increased expression of CCL2 in insulin-producing cells of transgenic mice promotes mobilization of myeloid cells from the bone marrow, marked insulitis, and diabetes. Diabetes 57: 3025-3033, 2008.
38. Newgard C B, McGarry J D. Metabolic coupling factors in pancreatic beta-cell signal transduction. Annu Rev Biochem 64: 689-719, 1995.
39. O'Neill C M, Lu C, Corbin K L, Sharma P R, Dula S B, Carter J D, Ramadan J W, Xin W, Lee J K, Nunemaker C S. Circulating levels of IL-1B+LL-6 cause ER stress and dysfunction in islets from prediabetic male mice. Endocrinology 154: 3077-3088, 2013.
40. Rees D D, Palmer R M, Schulz R, Hodson H F, Moncada S. Characterization of three inhibitors of endothelial nitric oxide synthase in vitro and in vivo. Br J Pharmacol 101: 746-752, 1990.
41. Rink J S, Chen X, Zhang X, Kaufman D B. Conditional and specific inhibition of NF-kappaB in mouse pancreatic beta cells prevents cytokine-induced deleterious effects and improves islet survival posttransplant. Surgery 151: 330-339, 2012.
42. Rink J S, McMahon K M, Zhang X, Chen X, Mirkin C A, Thaxton C S, Kaufman D B. Knockdown of intraislet IKKbeta by spherical nucleic acid conjugates prevents cytokine-induced injury and enhances graft survival. Transplantation 96: 877-884, 2013.
43. Roy I, Evans D B, Dwinell M B. Chemokines and chemokine receptors: update on utility and challenges for the clinician. Surgeiy 155: 961-973, 2014.
44. Sandler S, Andersson A, Hellerstrom C. hihibitory effects of interleukin 1 on insulin secretion, insulin biosynthesis, and oxidative metabolism of isolated rat pancreatic islets. Endocrinology 121: 1424-1431, 1987.
45. Sarkar S A, Lee C E, Victorino F, Nguyen I T, Walters J A, Burrack A, Eberlein J, Hildemann S K, Homann D. Expression and regulation of chemokines in murine and human type 1 diabetes. Diabetes 61: 436̂146, 2012.
46. Sehisler J C, Fueger P T, Babu D A, Hohmeier H E, Tessem J S, Lu D, Becker T C, Naziruddin B, Levy M, Mirmira R G, Newgard C B. Stimulation of human and rat islet beta-cell proliferation with retention of function by the homeodomain transcription factor Nkx6.1. Mol Cell Biol 28: 3465-3476, 2008.
47. Tapia-Limonchi R, Diaz I, Cahuana G M, Bautista M, Martin F, Soria B, Tejedo J R, Bedoya F J. Impact of exposure to low concentrations of nitric oxide on protein profile in murine and human pancreatic islet cells. Islets 6: e995997, 2014.
48. Tran W, Chen G, Newgard C B, Hohmeier H E. Discrete and complementary mechanisms of protection of beta-cells against cytokine-induced and oxidative damage achieved by bcl-2 overexpression and a cytokine selection strategy. Diabetes 52: 1423-1432, 2003.
49. Zhao G, Dharmadhikari G, Maedler K, Meyer-Hermann M. Possible role of interleukin-ip in type 2 diabetes onset and implications for anti-inflammatory therapy strategies. PLoS Comput Biol 10: el003798, 2014.

1. A method to indirectly assess insulin release in a cell in response to a test composition, the method comprising:
culturing pancreatic beta cells;
incubating the cells with a calcium sensitive indicator for an amount of time;
contacting the cells with the test composition;
stimulating the cells with glucose; and
measuring a signal from the calcium sensitive indicator.

2.-3. (canceled)

4. The method of claim 1, wherein the test composition is a pharmaceutical composition.

5. The method of claim 1, wherein the test composition is a chemical compound.

6. The method of claim 1, wherein the test composition is a biologic compound.

7. The method of claim 1, wherein the pancreatic beta cells are obtained from a subject in need thereof.

8. The method of claim 1, wherein the subject in need thereof has diabetes.

9. The method of claim 1, wherein the calcium sensitive indicator is a fluorescent indicator.

10. The method of claim 9, wherein the calcium sensitive indicator is selected from the group consisting of: fura-2, indo-1, fluo-3, fluo-4, calcium green-1, and a calcium binding fluorescent protein.

11. The method of any claim 1, wherein the step of measuring a signal comprises the step of microscopically visualizing the pancreatic beta cells.

12. The method of claim 11, wherein the step of measuring a signal further comprises detecting a wavelength of light emitted by the calcium sensitive indicator.

13. The method of claim 1, wherein the step of measuring a signal comprises detecting a wavelength of light emitted by the calcium sensitive indicator.

14. The method of claim 13, wherein the wavelength of light is detected by a plate reader.

15. The method of claim 1, wherein during the step of incubating the cells with a calcium sensitive indicator for an amount of time, the cells are also incubated with a surfactant.

16. A kit comprising:
a cell culture plate having a well;
pancreatic cells, where the pancreatic cells are contained in the well; and
a calcium sensitive indicator.

17. The kit of claim 16, wherein the calcium sensitive indicator is selected from the group consisting of: fura-2, indo-1, fluo-3, fluo-4, calcium green-1, and a calcium binding fluorescent protein.

18. The kit of claim 16, wherein the calcium sensitive indicator is contained in the well.

19. The kit of claim 1, wherein the calcium sensitive indicator is contained in the pancreatic cells.

20. The kit of claim 19, wherein the calcium sensitive indicator is a calcium binding fluorescent protein.
